(19) United States
(12) Patent Application Publication
(10) Pub. No.: US 2019/0062272 A1
(43) Pub. Date: Feb. 28, 2019

(54) SMALL MOLECULES FOR IMMUNOGENIC TREATMENT OF CANCER

(71) Applicant: Capten Therapeutics Inc., Woburn, MA (US)

(72) Inventors: Dinesh CHIMMANAMADA, Arlington, MA (US); Weiwen YING, Lexington, MA (US)

(73) Assignee: Capten Therapeutics Inc., Woburn, MA (US)

(21) Appl. No.: 16/093,294

(22) PCT Filed: Apr. 12, 2017
(86) PCT No.: PCT/US17/27248

§ 371 Date: Oct. 12, 2018

(51) Int. Cl. C07C 323/25; A61P 35/00; C07C 323/41; C07C 39/08; C07C 39/19; C07C 50/02

(52) U.S. Cl. CPC C07C 323/25; A61P 35/00; C07C 323/41; A61K 47/6883; C07C 39/19; C07C 50/02; C07C 39/08

(57) ABSTRACT

The present invention relates to new compounds for immunogenic treatment of cancer. The compounds can be administered as a single agent or in combination with an anticancer drug including modulators of other immune pathways, especially immune checkpoint inhibitors that target CTLA-4, PD-1, and PD-L1 proteins. The compounds can produce neoantigens through irreversible protein binding in cancer cells and generate immune response.

### TECHNICAL FIELD

The present invention relates to new compounds for immunogenic treatment of cancer. The compounds can be administered as a single agent or in combination with an anticancer drug including modulators of other immune pathways, especially immune checkpoint inhibitors that target CTLA-4, PD-1, and PD-L1 proteins. The compounds can produce neoantigens through irreversible protein binding in cancer cells and generate immune response.

### BACKGROUND OF THE INVENTION

Cancer immunotherapy has emerged as a breakthrough therapeutic area for the treatment of cancer. A significant population of melanoma and non-small cell lung cancer (NSCLC) patients have been proven to have benefited from antibody therapies to immune checkpoints such as cytotoxic lymphocyte antigen-4 (CTLA-4) and programmed cell death 1 (PD-1) (Hodi et al. N Engl J Med, 2010, 363, 711-723; Topalian et al. N Engl J Med, 2012, 366, 2443-2454). With one CTLA-4 and two PD-1 inhibitors approved by FDA for melanoma and NSCLC patients and recent reports of their successful application to other cancer types in trials, the future of cancer immune therapy looks brighter than ever. However, the percentage of population (overall response rate, ORR) who are benefited from these immune checkpoint inhibitors are relatively low, especially in types such as cervical cancer (Alexandrov et al., Nature, 2013, 500, 415-421) when given as single agents. Additionally, therapeutic response followed by development of resistance due to various mechanisms (Medler et al., Trends in Cancer, 2015, 1, 66-75) for the immune checkpoint inhibitors compel the researchers to find a way around it. Therefore, there are multiple strategies that are being followed, including combination therapies in the clinic, with various cytotoxic agents, molecularly targeted agents and other immune pathway modulators that are likely to be synergistic with the unblocking of these immune checkpoints.

Due to the low response rates that are being reported for these immune checkpoint inhibitors, several ways of enhancing immune responses are being considered. A report/review discusses about four different nodes of inducing or enhancing antitumor immunity (Smyth et al., Nat. Rev. Clin. Oncol, 2015, 24 Nov. 2015 online) namely; 1. Elimination of immune suppression; 2. Immunogenic cancer dell death; 3. Enhanced antigen presenting cell function; and 4. Enhanced T/macrophage effector activity. In the elimination of immune suppression category, multiple pathways/targets are currently being pursued such as IDO, Arginase-1, LAG-3, TIM-3, VISTA etc. In the APC function enhancement category, CD40, TLR agonists are being considered. Enhancing T or macrophage effector activity through modulation of tumor microenvironment (TME) is another emerging area with multiple targets such as proteins involved in adenosine pathway (A2AR, CD73), OX-40 and selective delivery of IL-2 are also being developed. However, the node 2 described by authors, which is immunogenic cancer cell death, offers unique opportunity to utilize various methodologies to generate artificial immunity at the tumor site. Vaccines to generate infiltration of cytotoxic lymphocytes (Melero et al., Nat. Rev. Clin. Oncol, 2014, 11, 509-524) are already underway or induction of chemokines to attract CTL are being conceived.

It has been reported that for the blockade of checkpoints such as PD-1 to be effective as anticancer strategy, there is a requisite of pre-existing antitumor CD8+ T-Cells that are negatively regulated by PD-1/PD-L1 mediated adaptive immune resistance (Tumeh et al., Nature, 2014, 515, 568-571). With the complex interplay of different immune cell populations among patients with progressive tumors, the impediment of antitumor immunity is difficult to overcome uniformly. Therefore, effective clinical management of antitumor immune responses requires careful consideration of synergistic mechanisms and manipulation of pathways or processes that would eventually lead to greater clinical outcome. One such process that would be synergistic with unblocking immune checkpoints is creation of immunogenic cell death (ICD) with the use of haptens or reactive chemical molecules that would generate neoantigens.

Chemically reactive small molecule drugs can be immunogenic, where hapten specific neoantigens are presented by MHC molecules to attract T cells leading to hypersensitivity or inflammation. Several modes of antigen presentations have been proposed (Pichler, Toxicology, 2002, 181-182, 49-54), irreversible binding of drugs/metabolites to intracellular proteins in targets organs generating antigens to attract T cells, direct binding to different T cell clones etc.

Haptens are small molecules that are too small to be recognized by the immune system, hence are not immunogenic. However, these reactive chemicals irreversibly bind to proteins creating antigenic epitopes, which in turn attract immune cells. The immunogenicity is determined by the reactivity of the hapten, hydrophobicity of the molecules and bioavailability (Chipinda et al., Journal of Allergy, vol. 2011, Article ID 839682, 11 pages, 2011.) Antigen presentation also depends on whether the irreversibly modified proteins are on the cell surface and cross presented through MHC-1 to cytotoxic CD8+ cells (Ortmann et al., The Journal of Immunology, 1992, 148, 1445-1450). And presentation of antigens to naïve T cells leads to the formation of hapten specific memory T cells that has the capability to become hapten specific effector T cells (CD4+ and CD8+) eliciting the damage at the site of action.

Erkes et al. classify all the attempts that has been done so far in the area of hapten-mediated antitumor immunity into 4 types (Erkes et al., Journal of Immunological Research, 2014, Article ID 175265, 28 pages): 1. Ex-vivo haptenation of the excised tumor and injecting the cells back to the animal (Hamoaka, et al., J. Exp. Medicine, 1979, 149, 185-189; Flood et al., J. of Immunol, 1987, 138, 3573-3579); 2. In situ generation of hapten (Fujiwara et al., The Journal of Immunology, 1984, 132, 1571-1577; Fujiwara et al., The Journal of Immunology, 1984, 133, 509-514); 3. Epifocal haptenation at the tumor site, practically achievable only for cutaneous tumors (Strobbe et al, Melanoma Research, vol. 7, no. 6, 507-512, 1997; Nida et al, Australasian Journal of Dermatology, vol. 44, no. 4, 277-280, 2003; Wack et al, Cancer Immunology, Immunotherapy, vol. 51, no. 8, pp. 431-439, 2002.). However, a recent publication also describes the use of trinitrophenol as the hapten along with an oxidant and combining with the drug Ara-c for the treatment of non-small cell lung cancer patients through local intratumoral delivery (Yu et al. Lung Cancer: Targets and Therapy, 2015, 6, 1-11); and 4. Administration of antigen-hapten conjugates to patients with antigen-receptor high tumors (Lu et al., Molecular Pharmaceutics, vol. 4, no. 5, pp. 695-706, 2007; Lu et al., and P. S. Low,” Cancer Immunology, Immunotherapy, vol. 51, no. 3, pp. 153-162, 2002). The following chemicals shown below are generally used for these studies (Lu et al., Int. J. Cancer, 2005, 116, 710-719 (2005))

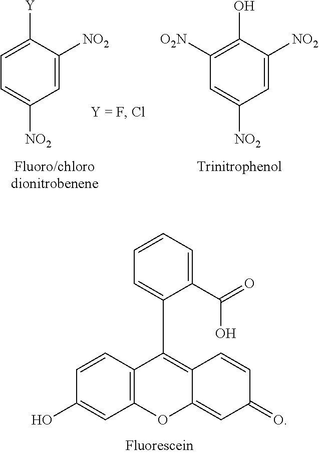

Urushiols, the chemicals that are found in plants such as poison oak, Lacquer Tree, poison ivy, poison sumac etc. are known to cause contact hypersensitivity (CHS) in people through the generation of immune response. It has been proposed that these catechol derivatives undergo oxidation to produce the quinones, which are highly reactive and bind to proteins irreversibly to generate neoantigens that attract immune response (Kalish et al., Journal of Allergy and Clinical Immunology, 1999, 103, 192-199; Ishii-Osai et al., Journal Dermatol Sci. 2012, 67, 51-60). Attempts of local application of urushiols to demonstrate anti-cancer effects have been reported (Goodman, U.S. Pat. No. 6,355,691; Goldberg, et al., PCT International. Application Publication No. WO 2000037067 A2; Martin, U.S. Patent Application Publication No. 20020001573 A1).

Here, a unique way is described to induce immunogenic cancer cell death through the utilization of chemically reactive small molecules as haptens that in turn generate neoantigens, attracting hapten specific cytotoxic T-cells. These neoantigen generating small molecules can either be used as a single agent or in combination with existing anticancer drugs, including modulators of other immune pathways, especially immune checkpoint inhibitors that target CTLA-4, PD-1, and PD-L1 proteins.

### SUMMARY OF THE INVENTION

In one aspect, the present invention provides a compound, or a pharmaceutically acceptable salt, conjugate, or prodrug thereof, wherein said compound is represented by Formula 1

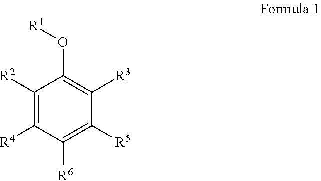

wherein
R¹ is H, PG, or PDG;
R² and R⁴ are each independently H, halogen, alkyl, haloalkyl, or aryl, wherein said alkyl, haloalkyl, and aryl are each optionally substituted with —NR⁷R^{7′}, —OR⁸, —SR⁸, or —SO₂—R⁸;
R³ is H, halogen, OH, —O-PG, —O-PDG, alkyl, haloalkyl, cycloalkyl, aryl, heteroaryl, —NR⁹R^{9′}; or R³ and R¹, together with the atoms to which they are attached, form a heterocyclic group;
R⁵ and R⁶ are independently H, halogen, OH, —OPG, —OPDG, alkyl, alkenyl, alkynyl, cycloalkyl, or heterocycloalkyl, wherein said alkyl, alkenyl, alkynyl, cycloalkyl, and heterocycloalkyl are each optionally substituted with NR⁹R^{9′} or OR^{10′}, and wherein when R⁵ is H, R⁶ is not H; and when R⁶ is H, R⁵ is not H; or
R⁵ and R⁶ each are independently selected from the group consisting of —S-alkyl, —S—(CH₂)ₚNR⁹R^{9′}, —S—(CH₂)ₚOR¹⁰, —O-alkyl, —O—(CH₂)ₚNR⁹R^{9′}, —O—(CH₂)ₚOR¹⁰, —NR¹¹R¹², —NR¹¹—(CH₂)ₚNR⁹R^{9′}, and —NR¹¹—(CH₂)ₚOR¹⁰;
R⁷, R^{7′}, R⁹, and R^{9′} are each independently H, alkyl, aryl, —CO-alkyl, —CO-cycloalkyl, —CO-aryl, —CO-heteroaryl, —SO₂-alkyl, —SO₂-cycloalkyl, —SO₂-aryl, or —SO₂-heterocycloalkyl, wherein said alkyl, cycloalkyl, heterocycloalkyl, aryl, and heteroaryl are each optionally substituted with a substituent independently selected from halo, amino, C₁₋₃ alkylamino, di-C₁₋₃ alkylamino, NO₂, C₁₋₅ alkyl, O—C₁₋₃ alkyl, cyano, C₁₋₃ haloalkyl, O—C₁₋₃ haloalkyl, COOH, cycloalkyl, heterocycloalkyl, aryl, and heteroaryl; or
R⁷ and R^{7′} or R⁹ and R^{9′}, together with the nitrogen atom to which they are attached, form a 4, 5, 6, or 7-membered heterocyclic ring;
R⁸ is alkyl, cycloalkyl, haloalkyl, aryl, or heteroaryl;
R¹⁰ is H, alkyl, haloalkyl, cycloalkyl, aryl, or heteroaryl;
R¹¹ is alkyl, aryl, or heteroaryl;
R¹² is H or alkyl;
p is an integer from 2 to 8;
PG is —PO₃H₂ or —SO₂NR^{m}Rⁿ;
R^{m} and Rⁿ are independently H, alkyl, or aryl; or R^{m} and Rⁿ, together with the nitrogen atom to which they are attached, form a 4, 5, 6, or 7-membered heterocyclic ring;
PDG is a prodrug moiety (PM) or a targeting moiety (TM), wherein said PDG group is connected to the group to which it is attached through a carbamate bond, an ester bond, or an ether bond.

In another aspect, the present invention provides a pharmaceutical composition comprising a compound of the invention as described herein, or a pharmaceutically acceptable salt, conjugate, or prodrug thereof, and one or more pharmaceutically acceptable carriers, diluents, and excipients.

In yet another aspect, the present invention provides a method for treating cancer, the method comprising the step of administering a therapeutically effective amount of a compound of the invention as described herein, or a pharmaceutically acceptable salt, conjugate, or prodrug thereof, or a pharmaceutical composition thereof, to a subject in need thereof, wherein the compound of the invention generates an immune response in situ at the tumor through a formation of neoantigen.

In some embodiments, the compound of the invention as described herein, or a pharmaceutically acceptable salt, conjugate, or prodrug thereof, or a pharmaceutical composition thereof, is administered in combination with an anticancer drug, wherein said anticancer drug is a cytotoxic drug selected from alkylating agents, antimitotic drugs, antimetabolites, topoisomerase 1 and 2 inhibitors, platinum drugs, anti-microtubule agents, and hormones, or derivatives thereof, a molecularly targeted agent, cell cycle signaling agent, modulators of tumor microenvironment, an antibody drug conjugate (ADC), radiation, an immunosuppressant, an enhancer of antigen presenting functions, an enhancer of T/macrophage effector, a drug that influences the purine metabolism, an agonist or antagonist of adenosine receptors, a proteasome inhibitor, a HDAC inhibitor, a TRAIL-R agonist, a chimeric antigen receptor t-cell therapy (CAR-T), an antitumor vaccine, or an immunomodulatory agent targeting PD-1, PD-L, NKG2A, KIR, CTLA-4, LAG-3, TIM-3, BTLA, VISTA, PD-1H, TIGIT, CD96, STAT3, Arginase-1, HIF-1a, VEGF, CCL2, IDO, Tie2, CSF1, IL-10, IL-13, or IL-23.

In other embodiments, the compound of the invention as described herein is administered in the form of:
(1) a prodrug,
(2) an antibody drug conjugate (ADC),
(3) a folate receptor drug conjugate (FRDC),
(4) a peptide drug conjugate,
(5) a nanoparticle containing liposomes, polymer based vehicles, or hyaluronic acid based delivery vehicles;
(6) a conjugate with a selective tyrosine kinase inhibitor; or
(7) a conjugate with an Hsp90 inhibitor.

The details of one or more embodiments of the invention are set forth in the accompanying the description below. Other features, objects, and advantages of the invention will be apparent from the description and drawings, and from the claims.

### BRIEF DESCRIPTION OF THE DRAWINGS

FIG. 1 depicts the effect of test compounds of the invention on IFN-γ production by mouse splenocytes.

FIG. 2 depicts the tumor growth inhibition by Compound 10 and synergy with PD-1 mAb in CT26 mouse syngeneic model.

FIG. 3 depicts the % Body weight change of animals treated with Compound 10 and PD-1 mAb in CT26 mouse syngeneic model.

FIG. 4 depicts % BW changes of the animals treated with vehicle and compound 10.

FIG. 5 depicts the frequency of T-cells.

FIG. 6 depicts the frequency of CD8 positive cells.

FIG. 7 depicts the frequency of CD4 positive T-Cells.

FIG. 8 depicts an immune response of Compound 10 as assessed by mouse ear swelling test (MEST).

FIG. 9 depicts the covalent bond formation of Compound 10 with cysteine in open air conditions.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### DETAILED DESCRIPTION OF THE PRESENT INVENTION

The present invention provides new compounds for immunogenic treatment of cancer.

In one aspect, the present invention provides a compound represented by Formula 1 The present invention provides a compound, or a pharmaceutically acceptable salt, a conjugate, or a prodrug thereof, wherein said compound is represented by Formula 1

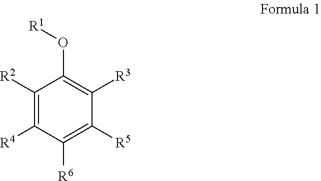

wherein
R¹ is H, PG, or PDG;
R² and R⁴ are each independently H, halogen, alkyl, haloalkyl, or aryl, wherein said alkyl, haloalkyl, and aryl are each optionally substituted with —NR⁷R^{7′}, —OR⁸, —SR⁸, or —SO₂—R⁸;
R³ is H, halogen, OH, —O-PG —O-PDG, alkyl, haloalkyl, cycloalkyl, aryl, heteroaryl, —NR⁹R⁹′; or R³ and R¹, together with the atoms to which they are attached, form a heterocyclic group;
R⁵ and R⁶ are independently H, halogen, OH, —OPG, —OPDG, alkyl, alkenyl, alkynyl, cycloalkyl, or heterocycloalkyl, wherein said alkyl, alkenyl, alkynyl, cycloalkyl, and heterocycloalkyl are each optionally substituted with NR⁹R^{9′} or OR^{10′}, and wherein when R⁵ is H, R⁶ is not H; and when R⁶ is H, R⁵ is not H; or R⁵ and R⁶ each are independently selected from the group consisting of —S-alkyl, —S—(CH₂)ₚNR⁹R^{9′}, —S—(CH₂)ₚOR¹⁰, —O-alkyl, —O—(CH₂)ₚNR⁹R^{9′}, —O—(CH₂)ₚOR¹⁰, —NR¹¹R¹², —NR¹¹—(CH₂)ₚNR⁹R^{9′}, and —NR¹¹—(CH₂)ₚOR¹⁰;
R⁷, R^{7′}, R⁹, and R^{9′} are each independently H, alkyl, aryl, —CO-alkyl, —CO-cycloalkyl, —CO-aryl, —CO-heteroaryl, —SO₂-alkyl, —SO₂-cycloalkyl, —SO₂-aryl, or —SO₂-heterocycloalkyl, wherein said alkyl, cycloalkyl, heterocycloalkyl, aryl, and heteroaryl are each optionally substituted with a substituent independently selected from halo, amino, C₁₋₃ alkylamino, di-C₁₋₃ alkylamino, NO₂, C₁₋₅ alkyl, O—C₁₋₃ alkyl, cyano, C₁₋₃ haloalkyl, O—C₁₋₃ haloalkyl, COOH, cycloalkyl, heterocycloalkyl, aryl, and heteroaryl; or
R⁷ and R^{7′} or R⁹ and R^{9′}, together with the nitrogen atom to which they are attached, form a 4, 5, 6, or 7-membered heterocyclic ring, optionally further substituted with an O, S, or N atom;
R⁸ is alkyl, cycloalkyl, haloalkyl, aryl, or heteroaryl;
R¹⁰ is H, alkyl, haloalkyl, cycloalkyl, aryl, or heteroaryl;
R¹¹ is alkyl, aryl, or heteroaryl;
R¹² is H or alkyl;
p is an integer from 2 to 8;
PG is —PO₃H₂ or —SO₂NR^{m}Rⁿ;
R^{m} and Rⁿ are independently H, alkyl, or aryl; or R^{m} and Rⁿ, together with the nitrogen atom to which they are attached, form a 4, 5, 6, or 7-membered heterocyclic ring;
PDG is a prodrug moiety (PM) or a targeting moiety (TM), wherein said PDG group is connected to the group to which it is attached through a carbamate bond, an ester bond, or an ether bond.

In some embodiments, in the compound of the invention, such as formula 1, the 4, 5, 6, or 7-membered ring formed by R⁷ and R^{7′} or R⁹ and R^{9′} is

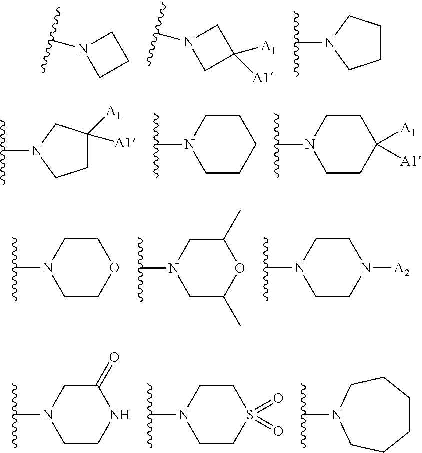

wherein
A₁ and A1′ are H, F, or CH₃;
A₂ is H, —CH₃, -Et, -nPr, -iPr, —C(O)—CH₃, —S(O)₂—CH₃, —S(O)₂-Et, —S(O)₂-nPr, or —S(O)₂-iPr.

In some embodiments, in the compound of the invention, such as formula 1, the targeting moiety (TM) is an antibody, a folate receptor binding moiety, a peptide, a nanoparticle based delivery vehicle, a selective tyrosine kinase inhibitor, or an Hsp90 inhibitor.

In some embodiments, in the compound of the invention, such as formula 1, the targeting moiety (TM) is a targeting moiety connected through a nitrogen atom (—N(TM)) or an oxygen atom (—O(TM)) of the targeting moiety.

In some embodiments, as used herein, —N(TM) is defined as below:

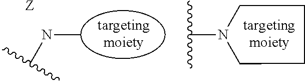

wherein the connecting nitrogen atom is either used as a link between the two fragment or part of the targeting moiety; and wherein Z is H, alkyl, heteroalkyl, aryl, heteroaryl, —OH, or —O— alkyl.

In some embodiments, if the targeting moiety in a compound of the invention as described herein is an antibody or other larger molecules, the linker includes, but is not limited to, a cathepsin cleavable valine-citruline linker with or without a spacer, or a disulfide linker, a phosphor-SPDB linker, a thioether linker, or a hydrazone/hydrazide linker.

In some embodiments, the targeting moiety includes (1) antibodies that are used in antibody drug conjugates including trastuzumab, gemtuzumab, inotuzumab, pinatuzumab, milatuzumab, lorvotuzumab, glembatumomab, labestuzumab, brentuximab, denintuzumab, anti-TROP-2; (2) folate receptor binding moieties such as folate and its derivatives; (3) peptides that used in targeted delivery such as LRP-1; (4) nanoparticle based delivery vehicles such as liposomes, polymer (such as polyethylene glycol) based delivery vehicles, hyaluronic acid based delivery vehicles.

In some embodiments, the targeting moiety can be a selective tyrosine kinase inhibitor. In some embodiments, the selective tyrosine kinase inhibitor can include a core structure of gefitinib, erlotinib, afatinib, canertinib, dacomitinib, neratinib, pelitinib, vandetanib, varlitinib, ibrutinib, acalabrutinib, ONO-4059, or spebrutinib.

In some embodiments, the PDG group in the compound of the invention as described herein is connected to the group to which it is attached through a carbamate bond, an ester bond, or an ether bond with an additional linker. In other embodiments, the PDG group in the compound of the invention is connected to the group to which it is attached through a carbamate bond, an ester bond, or an ether bond without an additional linker.

In some embodiments, in the compound of the invention, such as formula 1, PDG is represented by Formula 2, Formula 3, or Formula 4

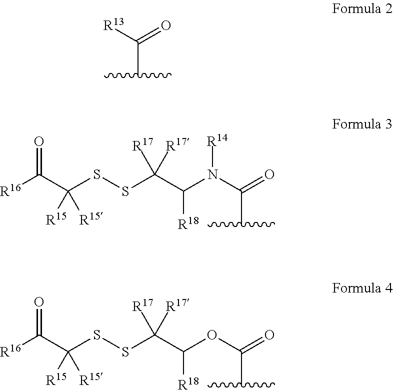

wherein
R¹³ is alkyl, haloalkyl, cycloalkyl, heterocycloalkyl, —NR⁹R^{9′}, —O-alkyl, or -(TM);
R¹⁴ is H, alkyl, haloalkyl, or cycloalkyl, wherein said alkyl, haloalkyl, and cycloalkyl contain 5-10 carbon atoms;
R¹⁵, R^{15′}, R¹⁷, or R^{17′} are each independently H, alkyl, or haloalkyl; or R¹⁵ and R^{15′} or R¹⁷ and R^{17′}, together with the carbon atom to which they are attached, form a 3, 4, 5-, or 6-membered heterocyclic ring;
R¹⁶ is —NR⁹R^{9′}, -TM, alkyl, haloalkyl, cycloalkyl, aryl, or heteroaryl; and
R¹⁸ is H or alkyl.

In some embodiments, the compound of Formula 1 is represented by Formula 5a or its oxidized quinone form of Formula 5b

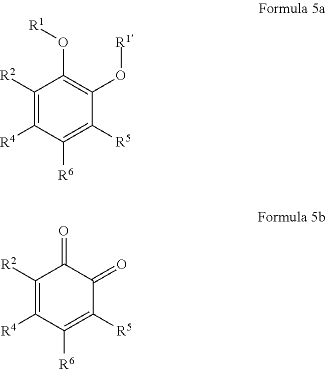

wherein
R⁶ is H, halogen, alkyl, haloalkyl, or cycloalkyl;
R^{1′} is H or PDG; or
R¹ and R^{1′}, together with the oxygen atoms to which they are attached, form a heterocyclic group represented by

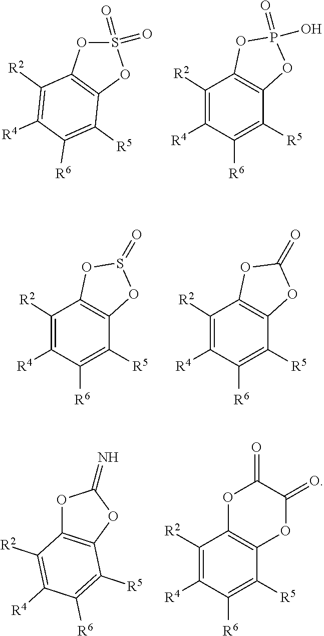

In some embodiments, the compound of Formula 1 is represented by Formula 6a or its oxidized quinone form of Formula 6b

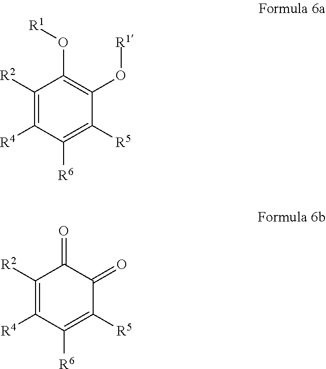

wherein
R⁵ is H, halogen, alkyl, haloalkyl, or cycloalkyl;
R^{1′} is H or PDG; or
R¹ and R^{1′}, together with the oxygen atoms to which they are attached, form a heterocyclic group represented by:

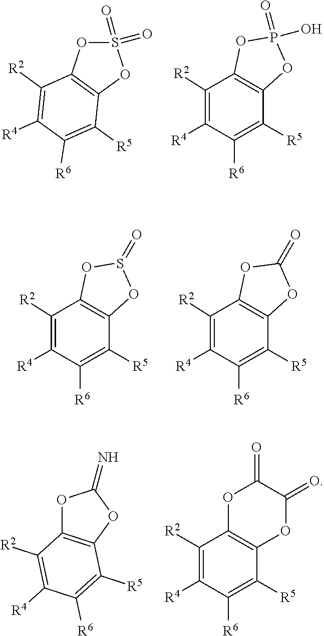

In some embodiments, the compound of Formula 1b is represented by Formula 7a or its oxidized quinone form of Formula 7b

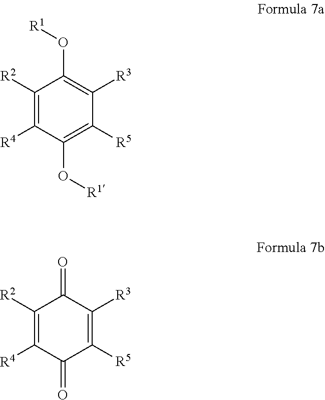

wherein R^{1′} is H or PDG.

In some embodiments, the compound of Formula 1 is represented by Formula 8a or its oxidized quinone form of Formula 8b

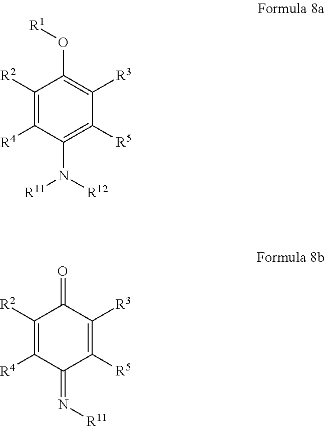

wherein
R¹¹ is aryl or heteroaryl, represented by the formulae:

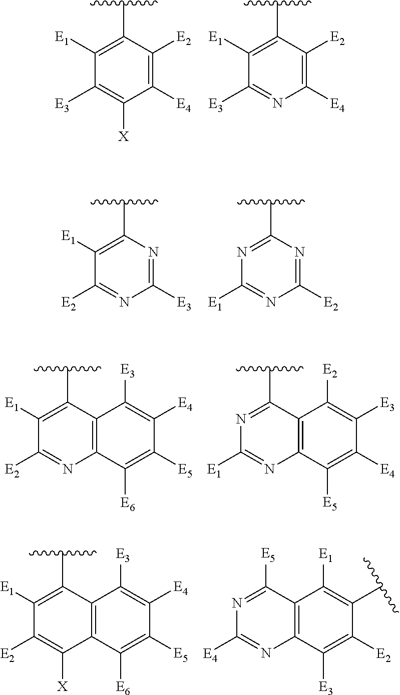

X is —O-alkyl or —NR^{m}Rⁿ;
E₁ to E₆ are each independently halo, —CH₃, -Et, —OCH₃, —OEt, or —CF₃;
R¹² is H; and
R^{m} and Rⁿ are independently H, alkyl, or aryl; or R^{m} and Rⁿ, together with the nitrogen atom to which they are attached, form a 4, 5, 6, or 7-membered heterocyclic ring;

In some embodiments, in the compound of the invention, for example, in the compound of Formulae 1, the 4, 5, 6, or 7-membered ring formed by R⁷ and R^{7′} or R⁹ and R^{9′} or R^{m} and Rⁿ is

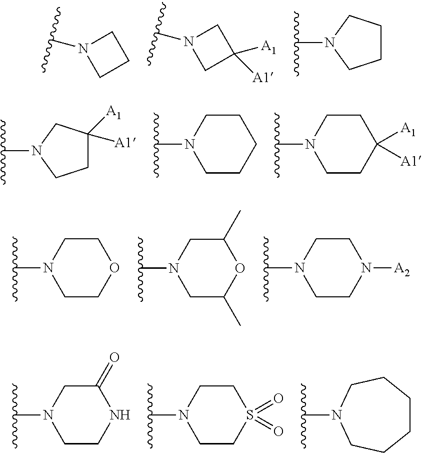

wherein
A₁ and A1′ are H, F, or CH₃;
A₂ is H, —CH₃, -Et, -nPr, -iPr, —C(O)—CH₃, —S(O)₂—CH₃, —S(O)₂-Et, —S(O)₂-nPr, or —S(O)₂-iPr.

In some embodiments, in the compound of the invention as described herein, for example, a compound of Formulae 1-4, 5a, 5b, 6a, 6b, 7a, 7b, 8a, and 8b, R¹, R², R⁴, and R⁵ are H. In some embodiments, R¹, R², R⁴, and R⁶ are H. In other embodiments, R¹, R², R³, and R⁴ are H. In certain embodiments, R¹, R³, R⁴, and R⁵ are H.

In some embodiments, in the compound of the invention as described herein, for example, a compound of Formulae 1-4, 5a, 5b, 6a, 6b, 7a, 7b, 8a, and 8b, R² is —CH₂—N(CH₃)₂, —CH₂—N(Et)₂, or —CH₂-Het.

In some embodiments, in the compound of the invention as described herein, for example, a compound of Formulae 1-4, 5a, 5b, 6a, 6b, 7a, 7b, 8a, and 8b, R³ is OH.

In some embodiments, in the compound of the invention as described herein, for example, a compound of Formulae 1-4, 5a, 5b, 6a, 6b, 7a, 7b, 8a, and 8b, R⁶ is OH.

In some embodiments, in the compound of the invention as described herein, for example, a compound of Formulae 1-4, 5a, 5b, 6a, 6b, 7a, 7b, 8a, and 8b, R⁶ is —S(CH₂)ₚNR⁹R^{9′} or —S—(CH₂)ₚOR¹⁰.

In some embodiments, in the compound of the invention as described herein, for example, a compound of Formulae 1-4, 5a, 5b, 6a, 6b, 7a, 7b, 8a, and 8b, p is an integer from 2 to 6. In certain embodiments, p is 2.

In some embodiments, in the compound of the invention as described herein, for example, a compound of Formulae 1-4, 5a, 5b, 6a, 6b, 7a, 7b, 8a, and 8b, wherein one of R⁹ and R^{9′} is H. In some embodiments, one of R⁹ and R^{9′} is —SO₂-alkyl, —SO₂-cycloalkyl, —SO₂-aryl, or —SO₂-heterocycloalkyl. In other embodiments, one of R⁹ and R^{9′} is —CO-alkyl, —CO-cycloalkyl, —CO-aryl, or —CO-heteroaryl. In some embodiments, one of R⁹ and R^{9′} is —CO-alkyl or —CO— cycloalkyl.

In some embodiments, in the compound of the invention as described herein, for example, a compound of Formulae 1-4, 5a, 5b, 6a, 6b, 7a, 7b, 8a, and 8b, R¹, R², R⁴ and R⁵ are H and R³ is OH. In some embodiments, R⁶ is alkenyl. In some embodiments, R⁶ is alkyl, —S—(CH₂)₂—N(H)—C(O)-alkyl, or —S—(CH₂)₂—N(H)—C(O)-cycloalkyl. In some embodiments, the alkyl group, including the alkyl group from —S—(CH₂)₂—N(H)—C(O)-alkyl, is methyl, ethyl, n-propyl, i-propyl, n-butyl, sec-butyl, t-butyl, n-pentyl, n-hexyl, n-heptyl, n-octyl, n-nonyl, or n-decyl. In some embodiments, the alkyl group is methyl. In certain embodiments, the alkyl group is ethyl. In other embodiments, the cycloalkyl group, including the cycloalkyl group from —S—(CH₂)₂—N(H)—C(O)-cycloalkyl, is -cyclopropyl, -cyclobutyl, -cyclopentyl, -cyclohexyl, or -cycloheptyl. In certain embodiments, alkyl is optionally substituted with halogen. In some embodiments, the optionally substituted alkyl is trifluoromethyl.

In some embodiments, in the compound of the invention as described herein, for example, a compound of Formulae 1-4, 5a, 5b, 6a, 6b, 7a, 7b, 8a, and 8b, R¹, R², R⁴ and R⁶ are H and R³ is OH. In some embodiments, R⁵ is alkyl, alkenyl, or alkynyl, wherein each of alkyl, alkenyl, and alkynyl is a linear hydrocarbon group containing 2-20 carbon atoms. In other embodiments, R⁵ is cycloalkyl. In some embodiments, R⁵ is —S—(CH₂)₂—N(H)—C(O)-alkyl or —S—(CH₂)₂—N(H)—C(O)-cycloalkyl. In some embodiments, the alkyl group, including the alkyl group from —S—(CH₂)₂—N(H)—C(O)-alkyl, is methyl, ethyl, n-propyl, i-propyl, n-butyl, sec-butyl, t-butyl, n-pentyl, n-hexyl, n-heptyl, n-octyl, n-nonyl, or n-decyl. In some embodiments, the alkyl group is methyl. In certain embodiments, the alkyl group is ethyl. In other embodiments, the cycloalkyl group, including the cycloalkyl group from —S—(CH₂)₂—N(H)—C(O)-cycloalkyl, is -cyclopropyl, -cyclobutyl, -cyclopentyl, -cyclohexyl, or -cycloheptyl. In certain embodiments, alkyl is optionally substituted with halogen. In some embodiments, the optionally substituted alkyl is trifluoromethyl.

In some embodiments, in the compound of the invention as described herein, for example, a compound of Formulae 1-4, 5a, 5b, 6a, 6b, 7a, 7b, 8a, and 8b, R¹, R², R³ and R⁴ are H and R⁶ is OH. In some embodiments, R⁵ is alkenyl. In some embodiments, R⁵ is alkyl, cycloalkyl, —S—(CH₂)₂—N(H)—C(O)-alkyl, or —S—(CH₂)₂—N(H)—C(O)-cycloalkyl. In some embodiments, the alkyl group, including the alkyl group from —S—(CH₂)₂—N(H)—C(O)-alkyl, is methyl, ethyl, n-propyl, i-propyl, n-butyl, sec-butyl, t-butyl, n-pentyl, n-hexyl, n-heptyl, n-octyl, n-nonyl, or n-decyl. In some embodiments, the alkyl group is methyl. In certain embodiments, the alkyl group is ethyl. In other embodiments, the cycloalkyl group, including the cycloalkyl group from —S—(CH₂)₂—N(H)—C(O)-cycloalkyl, is -cyclopropyl, -cyclobutyl, -cyclopentyl, -cyclohexyl, or -cycloheptyl. In certain embodiments, alkyl is optionally substituted with halogen. In some embodiments, the optionally substituted alkyl is trifluoromethyl.

In some embodiments, in the compound of the invention as described herein, for example, a compound of Formulae 1-4, 5a, 5b, 6a, 6b, 7a, 7b, 8a, and 8b, R¹, R³, R⁴ and R⁵ are H. In some embodiments, R² is —CH₂—N(CH₃)₂, —CH₂—N(Et)₂, or —CH₂-Het. In some embodiments, Het is azitidine, pyrrolidine, morpholine, piperidine, N-methylpiperazine, N-ethylpiperazine connected through nitrogen. In some embodiments, R⁶ is 7-chloroquinolin-4-amine, 7-methylquinolin-4-amine, or 7-trifluoromethylquinoline-4-amine. In certain embodiments, R⁶ is connected to other groups through the amino group at the 4′ position of optionally substituted pyridine or quinoline fragments.

In some embodiments, in the compound of the invention as described herein, for example, a compound of Formulae 1-4, 5a, 5b, 6a, 6b, 7a, 7b, 8a, and 8b, R¹ is —PO₃H₂, —SO₂—NH₂, —SO₂—N(CH₃)₂, —SO₂—N(Et)₂, —SO₂-Het, -acetyl, —CO—N(CH₃)₂, —CO—N(Et)₂, —CO—N(iPr)₂, —CO—N(nPr)₂, and —CO-Het. In some embodiments, Het is azitidine, pyrrolidine, morpholine, piperidine, N-methylpiperazine, or N-ethylpiperazine. In certain embodiments, Het is connected to other groups through its nitrogen atom.

In some embodiments, in the compound of the invention as described herein, for example, a compound of Formulae 1-4, 5a, 5b, 6a, 6b, 7a, 7b, 8a, and 8b, R³ is —O—PO₃H₂, —O—SO₂—NH₂, —O—SO₂—N(CH₃)₂, —O—SO₂—N(Et)₂ —O—SO₂-Het, —OAc, —O—CO—N(CH₃)₂, —O—CO—N(Et)₂, —O—CO—N(iPr)₂, —O—CO—N(nPr)₂, or —O—CO-Het. In some embodiments, Het is azitidine, pyrrolidine, morpholine, piperidine, N-methylpiperazine, or N-ethylpiperazine. In certain embodiments, Het is connected to other groups through its nitrogen atom.

In some embodiments, in the compound of the invention as described herein, for example, a compound of Formulae 1-4, 5a, 5b, 6a, 6b, 7a, 7b, 8a, and 8b, R⁶ is —O—PO₃H₂, —O—SO₂—NH₂, —O—SO₂—N(CH₃)₂, —O—SO₂—N(Et)₂ —O—SO₂-Het, —OAc, —O—CO—N(CH₃)₂, —O—CO—N(Et)₂, —O—CO—N(iPr)₂, —O—CO—N(nPr)₂, or —O—CO-Het. In some embodiments, Het is azitidine, pyrrolidine, morpholine, piperidine, N-methylpiperazine, or N-ethylpiperazine. In certain embodiments, Het is connected to other groups through its nitrogen atom.

In some embodiments, in the compound of the invention as described herein, for example, a compound of Formulae 1-4, 5a, 5b, 6a, 6b, 7a, 7b, 8a, and 8b, R¹ and R³, together with the atoms to which they are attached, form a 5- or 6-membered heterocyclic ring. In some embodiments, the 5- or 6-membered heterocyclic ring is a cyclic sulfite, a cyclic sulfate, a cyclic phosphate, a cyclic carbonate, a cyclic imidate, or a cyclic oxalate.

In some embodiments, as used herein, the term “conjugate” refers to a product of the covalent attachment of a compound of the invention as described herein, for example, a compound of formula 1, to an antibody, a folate receptor binding moiety, a peptide, a nanoparticle based delivery vehicle, a selective tyrosine kinase inhibitor, or an Hsp90 inhibitor.

In some embodiments, the conjugate of a compound of the invention as described herein is an antibody drug conjugate (ADC), a folate receptor drug conjugate (FRDC), a peptide drug conjugate, a nanoparticle containing liposomes, a polymer based vehicle, a hyaluronic acid based delivery vehicle, a conjugate with a selective tyrosine kinase inhibitor; or a conjugate with an Hsp90 inhibitor thereof.

In some embodiments, the compound of the invention is
N-(2-((3,4-dimethoxyphenyl)thio)ethyl)propionamide;
N-[2-(3,4-dihydroxyphenyl)sulfanylethyl]propenamide;
N-[2-(2,5-dihydroxyphenyl)sulfanylethyl]propenamide;
N-[2-(2,3-dihydroxyphenyl)sulfanylethyl]propenamide;
4-(2-aminoethylsulfanyl)benzene-1,2-diol;
N-[2-(3,4-dimethoxyphenyl)sulfanylethyl]heptanamide;
N-[2-(3,4-dihydroxyphenyl) sulfanylethyl]heptanamide;
N-[2-(3,4-dihydroxyphenyl) sulfanylethyl]undecanamide;
N-[2-(3,4-dimethoxyphenyl)sulfanylethyl]undecanamide;
N-[2-(3,4-dihydroxyphenyl) sulfanylethyl]undecanamide;
N-[2-(3,4-dimethoxyphenyl)sulfanylethyl]undecanamide;
N-[2-(3,4-dihydroxyphenyl) sulfanylethyl]pentanamide;
3-(5,5-difluoro-1,3-dimethyl-dipyrrolo[3,1-c:2′,1′-g][1,3,2]diazaborinin-4-ium-7-yl)-N-[2-(3,4-dihydroxyphenyl) sulfanylethyl]propenamide;
N-[2-(3,4-dihydroxyphenyl) sulfanylethyl]cyclopentanecarboxamide;
3-pentadecylbenzene-1,2-diol;
(4Z)-4-[(7-chloro-4-quinolyl)imino]-2-(diethylaminomethyl)cyclohexa-2,5-dien-1-one; or
3-[(Z)-pentadec-10-enyl]benzene-1,2-diol.

As used herein, the term “alkyl” refers to a saturated, straight or branched, noncyclic hydrocarbon having 1-25 carbon atoms. In some embodiments, the alkyl group contains 1-20 carbon atoms. In some embodiments, an alkyl group can contain from 1 to 25, from 1 to 20, from 2 to 20, from 1 to 10, from 1 to 8, from 1 to 6, from 1 to 4, or from 1 to about 3 carbon atoms. In other embodiments, exemplary alkyl groups include, but are not limited to, methyl, ethyl, n-propyl, isopropyl, butyl, sec-butyl, t-butyl, hexyl, and c-hexyl.

As used herein, the term “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of cyclopentane, cyclopentene, cyclohexane, and the like. A cycloalkyl group having one or more fused aromatic rings can be attached through either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like.

As used herein, the term “alkenyl” refers to an unsaturated, straight or branched, cyclic or noncyclic hydrocarbon group having 2-20 carbon atoms. Exemplary groups include, but are not limited to, —(CH₂)₇CH═CH(CH₂)SCH₃, —(CH₂)₇CH═CHCH₂CH═CH(CH₂)₂CH₃, (CH₂)₇CH═CHCH₂CH═CHCH═CHCH₃, (CH₂)₇CH═CHCH₂CH═CHCH₂CH═CH₂, or —(CH₂)₉CH═CH(CH₂)₃CH₃ [(Z)-3-(10-Pentadecenyl)-1,2-benzenediol].

As used herein, the term “alkynyl” refers to an unsaturated, straight or branched, noncyclic hydrocarbon group having 2-10 carbon atoms and containing one or more triple bonds.

In some embodiments, “halo” or “halogen” includes fluoro, chloro, bromo, and iodo.

As used herein, the term “haloalkyl” refers to an alkyl group functionalized with one or more halo groups. In other embodiments, the halo group is a fluoro group, or a chloro group, or a bromo group. In some embodiments, the halo group is a fluoro group. When the halo group is fluoro, exemplary haloalkyl includes, is not limited to, —CF₃, —CHF₂, —CH₃—CF₃, and —CF₂—CF₃.

As used herein, “heterocycloalkyl” or “heterocylic group” or “heterocyclic ring” or “Het” refers to a non-aromatic heterocycle where one or more of the ring-forming atoms is a heteroatom such as an O, N, and/or S atom. Heterocycloalkyl groups can include mono- or polycyclic (e.g., having 2, 3 or 4 fused rings) ring systems as well as spirocycles. Example heterocycloalkyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles. In some embodiments, the heterocycloalkyl group refers to piperidine, morpholine, pyrrolidine, piperazine, azetidine, or tetrahydrofuran. In some embodiments, the heterocycloalkyl group refers to

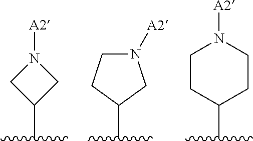

wherein A2′ is H, —CH₃, -Et, -nPr, or -iPr.

As used herein, “halo” or “halogen” includes fluoro, chloro, bromo, and iodo.

As used herein, the term “aryl” refers to monocyclic or polycyclic (e.g., having 2, 3 or 4 fused rings) aromatic hydrocarbons such as, for example, phenyl, naphthyl, and the like. In some embodiments, an aryl group has from 6 to about 20 carbon atoms. In other embodiments, an aryl group has from 6 to 10 carbon atoms. In some embodiments, the aryl group refers to phenyl or naphthyl.

In some embodiments, the term “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. In some embodiments, the heteroaryl group has from about 3 to about 20 carbon atoms. In some embodiments, the heteroaryl group contains 3 to 14, 3 to 7, or 5 to 6 ring-forming atoms. In some embodiments, the heteroaryl group has 1 to 4, 1 to 3, or 1 to 2 heteroatoms. Exemplary heteroaryl groups include, but are not limited to, furan, thiophene, pyrrole, pyridine, imidazole, pyrimidine, triazine, pyrazole, triazole, oxazole, indole, indazole, imidazole, indolizine, purine, indolizine, phthalazine, quinolone, isoquinoline, quinazoline, triazine, pyridazine, benzofuran, benzimidazole, oxadiazole, thiadiazole, isothiazole, tetrazole, and the like. In some embodiments, the heteroaryl group refers to pyrrole, furan, thiophene, thiazole, oxazole, pyrazole, imidazole, isoxazole, triazole, pyrimidine, pyridine, triazine, or pyrazine

As used herein, the term “compound,” as used herein, is meant to include all stereoisomers, geometric isomers, tautomers, and isotopomers of the structures depicted. All compounds are also meant to include solvated, or hydrated forms.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

In the case of the compounds which contain an asymmetric carbon atom, the invention relates to the D form, the L form, and D/L mixtures and also, where more than one asymmetric carbon atom is present, to the diastereomeric forms. Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

The compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the compound of the invention as described herein can be in different isotope variation, for example, isotopes of hydrogen can be tritium or deuterium.

In some embodiments, the compound of the invention as described herein can be in different polymorph forms. The term “polymorph” refers to a specific form of a compound of the invention. For example, polymorphs may represent crystalline forms that can vary in pharmaceutically relevant physical properties between one form and another, e.g., under different crystallization conditions, environmental conditions, hygroscopic activity of the compounds.

In some embodiments, the term “hydrate” includes, but is not limited to, hemi-hydrate, monohydrate, dihydrate, trihydrate and the like. Hydrates of a compound of the invention may be prepared by contacting the compound with water under suitable conditions to produce the hydrate of choice.

The present invention includes a pharmaceutically acceptable salt of the compounds described herein. As used herein, a “pharmaceutically acceptable salt” refers to derivatives of the disclosed compounds wherein the parent compound is modified by converting an existing acid or base moiety to its salt form. Examples of a pharmaceutically acceptable salt include, but are not limited to, mineral or organic acid salts of basic residues such as amines; alkali or organic salts of acidic residues such as carboxylic acids; and the like. The pharmaceutically acceptable salt of the present invention includes the conventional non-toxic salts of the parent compound formed, for example, from non-toxic inorganic or organic acids. The pharmaceutically acceptable salt of the present invention can be synthesized from the parent compound which contains a basic or acidic moiety by conventional chemical methods. Such salt can be prepared by reacting the free acid or base forms of these compounds with a stoichiometric amount of the appropriate base or acid in water or in an organic solvent, or in a mixture of the two. In some embodiments, nonaqueous media like ether, ethyl acetate, ethanol, isopropanol, or acetonitrile are used.

The physiologically acceptable salt may be obtained by neutralizing the bases with inorganic or organic acids or by neutralizing the acids with inorganic or organic bases. Examples of suitable inorganic acids are hydrochloric acid, sulphuric acid, phosphoric acid, or hydrobromic acid, while examples of suitable organic acids are carboxylic acid, sulpho acid, or sulphonic acid, such as acetic acid, tartaric acid, lactic acid, propionic acid, glycolic acid, malonic acid, maleic acid, fumaric acid, tannic acid, succinic acid, alginic acid, benzoic acid, 2-phenoxybenzoic acid, 2-acetoxybenzoic acid, cinnamic acid, mandelic acid, citric acid, maleic acid, salicylic acid, 3-aminosalicylic acid, ascorbic acid, embonic acid, nicotinic acid, isonicotinic acid, oxalic acid, gluconic acid, amino acids, methanesulphonic acid, ethanesulphonic acid, 2-hydroxyethanesulphonic acid, ethane-1,2-disulphonic acid, benzenesulphonic acid, 4-methylbenzenesulphonic acid or naphthalene-2-sulphonic acid. Examples of suitable inorganic bases are sodium hydroxide, potassium hydroxide and ammonia, while examples of suitable organic bases are amines, e.g., tertiary amines, such as trimethylamine, triethylamine, pyridine, N,N-dimethylaniline, quinoline, isoquinoline, α-picoline, β-picoline, γ-picoline, quinaldine, or pyrimidine.

In some embodiments, a physiologically acceptable salt of the compounds of the present invention can be obtained by converting derivatives which possess tertiary amino groups into the corresponding quaternary ammonium salts in a manner known per se using quaternizing agents. Examples of suitable quaternizing agents are alkyl halides, such as methyl iodide, ethyl bromide, and n-propyl chloride, and also arylalkyl halides, such as benzyl chloride or 2-phenylethyl bromide.

In some embodiments, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In some embodiments, a “therapeutically effective amount” as used herein refers to the amount which provides a therapeutic effect for a given condition and administration regimen.

The “subject” is used here to refer to an animal or a human. In some embodiment, the term “subject” refers to a human.

### Compositions and Administration

In another aspect, the present invention provides a pharmaceutical composition comprising a compound of the invention, e.g., a compound of Formulae 1-4, 5a, 5b, 6a, 6b, 7a, 7b, 8a, and 8b, or a pharmaceutically acceptable salt, conjugate, or prodrug thereof, and one or more pharmaceutically acceptable carriers, diluents, and excipients.

Pharmaceutically acceptable carriers include solvents, dispersion media, buffers, coatings, antibacterial and antifungal agents, wetting agents, preservatives, buffers, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof. Antibacterial and antifungal agents include parabens, chlorobutanol, phenol, ascorbic acid, and thimerosal.

The pharmaceutical compositions of the invention may be formulated in a variety of ways, including for example, solid, semi-solid (e.g., cream, ointment, and gel), and liquid dosage forms, such as liquid solutions (e.g., topical lotion or spray), dispersions or suspensions, tablets, pills, powders, liposomes, micelles, nanoparticles and suppositories. In some embodiments, the compositions are in the form of injectable or infusible solutions. The composition is in a form suitable for oral, intravenous, intraarterial, intramuscular, subcutaneous, parenteral, transmucosal, transdermal, or topical administration. The composition may be formulated as an immediate, controlled, extended or delayed release composition.

More particularly, pharmaceutical compositions suitable for use include sterile aqueous solutions (where water soluble) or dispersions and sterile powders for the extemporaneous preparation of sterile solutions or dispersions. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Sterile solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in U.S. Patent Application Publication No. 2002/0102208 A1, which is incorporated herein by reference in its entirety.

Effective doses of the compositions of the present invention, for the methods described herein vary depending upon many different factors, including means of administration, target site, physiological state of the subject, whether the subject is human or an animal, other medications administered, and whether treatment is prophylactic or therapeutic. Usually, the patient is a human but non-human mammals (e.g., domestic animals) can also be diagnosed or treated. In one embodiment, the methods of treatment described herein can be used to treat any suitable mammal, including primates, such as monkeys and humans, horses, cows, cats, dogs, rabbits, and rodents such as rats and mice. Diagnostic of treatment dosages may be titrated using routine methods known to those of skill in the art to optimize safety and efficacy.

The pharmaceutical compositions of the invention may include a “therapeutically effective amount” or a “diagnostically effective amount.” A “therapeutically effective amount” or a “diagnostically effective amount” refers to an amount effective, at dosages and for periods of time necessary, to achieve the desired therapeutic or diagnostic result, respectively. An effective amount may vary according to factors such as the disease state, age, sex, and weight of the individual, and the ability of the compound to elicit a desired response in the individual. An effective amount is also one in which any toxic or detrimental effects of the compound are outweighed by the therapeutically or diagnostically beneficial effects.

“Administration” or “administering” to a subject is not limited to any particular delivery system and may include, without limitation, oral (for example, in capsules, suspensions or tablets), parenteral (including subcutaneous, intravenous, intramedullary, intraarticular, intramuscular, or intraperitoneal injection), rectal, topical, and transdermal. Administration to a subject may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, e.g., Remington's Pharmaceutical Sciences, Mack Publishing Co.).

The dose of the active compound of the invention can vary depending on the route of administration, the age and weight of the patient, the nature and severity of the diseases to be treated, and similar factors. The daily dose can be given as a single dose, which is to be administered once, or be subdivided into two or more daily doses, and is as a rule 0.001-2000 mg. Particular preference is given to administering daily doses of 0.1-500 mg, e.g. 0.1-100 mg.

Suitable administration forms are oral, parenteral, intravenous, transdermal, topical, inhalative, intranasal and sublingual preparations. In some embodiments, administration forms are by parenteral, e.g. intravenous, intraperitoneal, or intramuscular.

As indicated above, the compound of the invention may be administered as a combination therapy with further active agents, e.g. therapeutically active compounds useful in the treatment of cancer, for example, prostate cancer, ovarian cancer, lung cancer, or breast cancer. For a combination therapy, the active ingredients may be formulated as compositions containing several active ingredients in a single dose form and/or as kits containing individual active ingredients in separate dose forms. The active ingredients used in combination therapy may be coadministered or administered separately.

### Pharmaceutical Methods

The present invention provides a method of selective tumor delivery and in situ generation of molecules that react with proteins to generate neoantigens at tumor, that attract specific immune cells such as CD8+, CD4+ cells. Such immune response generating chemicals through neoantigen formation can be used as single agents or their combination with other anticancer agents, especially modulators of other immune pathways including immune checkpoint inhibitors that target CTLA-4, PD-1, and PD-L1.

The present invention provides a method for treating cancer, the method comprising the step of administering to a subject in need thereof a therapeutically effective amount of a compound of the invention, for example, a compound of Formulae 1-4, 5a, 5b, 6a, 6b, 7a, 7b, 8a, and 8b, or a pharmaceutically acceptable salt, conjugate, or prodrug thereof. It is an embodiment of the invention that the compound of the invention generates an immune response in situ at the tumor through a formation of neoantigen. In some embodiments, the compound of the invention can be used for the treatment of cancer, for example, prostate cancer, ovarian cancer, lung cancer, breast cancer, melanoma, head and neck cancer, non-Hodgkin's lymphoma, bladder cancer, and chronic lymphocytic leukemia.

In some embodiments, the compound of the invention that are represented by Formulae 1-4, 5a, 5b, 6a, 6b, 7a, 7b, 8a, and 8b are specifically used for treating cancers that express elevated levels of reactive oxygen species. Such cancers include, but are not limited to, lung cancer, breast cancer, liver cancer, leukemia, uroepithelial cancer, pancreatic cancer, ovarian cancer, colorectal cancer, head and neck cancer, melanoma, esophageal cancer, glioblastoma, prostate cancer, oral cancer, bladder cancer, gastric cancer, cervical cancer, colon cancer, and others. Such applications are envisioned based on the idea that selective conversion of prohapten to hapten is feasible by reactive oxygen species within tumor microenvironment of the cancers.

In some embodiments, the compound of the invention such as a compound of Formulae 1-4, 5a, 5b, 6a, 6b, 7a, 7b, 8a, and 8b is administered in combination with an anticancer drug, wherein the anticancer drug is a cytotoxic drug selected from alkylating agents, antimitotic drugs, antimetabolites, topoisomerase 1 and 2 inhibitors, platinum drugs, anti-microtubule agents, and hormones, or derivatives thereof, a molecularly targeted agent, cell cycle signaling agent, modulators of tumor microenvironment, an antibody drug conjugate (ADC), radiation, an immunosuppressant, an enhancer of antigen presenting functions, an enhancer of T/macrophage effector, a drug that influences the purine metabolism, an agonist or antagonist of adenosine receptors, a proteasome inhibitor, a HDAC inhibitor, a TRAIL-R agonist, a chimeric antigen receptor t-cell therapy (CAR-T), an antitumor vaccine, or an immunomodulatory agent targeting PD-1, PD-L1, NKG2A, KIR, CTLA-4, LAG-3, TIM-3, BTLA, VISTA, PD-1H, TIGIT, CD96, STAT3, Arginase-1, HIF-1a, VEGF, CCL2, IDO, Tie2, CSF1, IL-10, IL-13, or IL-23.

In some embodiments, the anticancer drug used in combination with the compound of the invention for the treatment of cancer can be an immunomodulatory agent targeting CTLA-4, PD-1, or PD-L1.

In some embodiments, the cytotoxic drug can be anthracyclines, oxaliplatin, cisplatin, taxanes, or radiotherapy. In some embodiments, the enhancer of an antigen presenting function can be a SIRPa antagonist, a TLR agonist, or a CD40 agonist. In some embodiments, the enhancer of a T/macrophage effector can be a drug that agonizes a receptor such as CD28, CD28H, CD30, CD137, CD27, DNAM-1, HVEM, GITR, OX40, or ICOS. In some embodiments, the drug that influences the purine metabolism can be CD73 or CD39. In some embodiments, the agonist and antagonist of adenosine receptors can be A1AR, A2AR, A2BR, or A3AR.

In other embodiments, the compound of the invention as described herein, or is administered in the form of:
(1) a prodrug,
(2) an antibody drug conjugate (ADC),
(3) a folate receptor drug conjugate (FRDC),
(4) a peptide drug conjugate,
(5) a nanoparticle containing liposomes, polymer based vehicles, or hyaluronic acid based delivery vehicles;
(6) a conjugate with a selective tyrosine kinase inhibitor; or
(7) a conjugate with an Hsp90 inhibitor

As used herein, the term “prodrug” refers to a molecule that includes an active drug and in addition carries a moiety intended for a specific purpose. The specific purpose can be, a moiety added for making the compound or active drug soluble for administration, for selective cleavage at the site of action, to improve the pharmacokinetic profile of the parent drug. In some embodiments, simple chemical fragments, antibodies (ADC), Folic acid derivatives (FRDC), Hsp90 inhibitors and polymer such as PEG (PDC) can be part of the prodrug moieties.

As used herein, the term “modulators of other immune pathways” refer to drugs that eliminate immune suppression targeting, PD-1, PD-L1, NKG2A, KIR, CTLA-4, LAG-3, TIM-3, BTLA, VISTA, PD-1H, TIGIT, CD96, STAT3, Arginase-1, HIF-1a, VEGF, CCL2, IDO, Tie2, CSF1, IL-10, IL-13, IL-23; those drugs that induce immunogenic cancer death including; proteasome inhibitors, HDAC inhibitors, TRAIL-R agonists, cytotoxic drugs such as anthracyclines, oxaliplatin, cisplatin, taxanes, radiotherapy; enhancers of antigen presenting functions including SIRPa antagonists, TLR agonists, CD40 agonists; enhancers of T/macrophage effector activities including drugs that agonize receptors such as CD28, CD28H, CD30, CD137, CD27, DNAM-1, HVEM, GITR, OX40, ICOS, drugs that influence the purine metabolism such as CD73, CD39, agonists and antagonists of adenosine receptors such as A1AR, A2AR, A2BR, A3AR.

The present invention further provides a method for treating cancer, the method comprising the step of administering to a subject in need thereof a therapeutically effective amount of a compound of 2,4-dinitrochlorobenzene, 2,4-dinitrofluorobenzene, 2,4,6-trinitrobenzenesulfonic acid, 2,4,6-trinitrophenol, fluorescein, penicillin, flucloxacillin, trimethoprim, abacavir, lamotrigine, clozapine, and isoniazid, or a pharmaceutically acceptable salt, conjugate, or prodrug, or composition thereof. In the method of treating cancer of the present invention, a compound of 2,4-dinitrochlorobenzene, 2,4-dinitrofluorobenzene, 2,4,6-trinitrobenzenesulfonic acid, 2,4,6-trinitrophenol, fluorescein, penicillin, flucloxacillin, trimethoprim, abacavir, lamotrigine, clozapine, and/or isoniazid generates an immune response in situ at the tumor through a formation of neoantigen.

In some embodiments, in the method of treating cancer of the present invention, a compound of 2,4-dinitrochlorobenzene, 2,4-dinitrofluorobenzene, 2,4,6-trinitrobenzenesulfonic acid, 2,4,6-trinitrophenol, fluorescein, penicillin, flucloxacillin, trimethoprim, abacavir, lamotrigine, clozapine, and isoniazid, or a pharmaceutically acceptable salt, conjugate, or prodrug, or composition thereof, is administered in combination with another anticancer drug and wherein said anticancer drug is a cytotoxic drug selected from alkylating agents, antimitotic drugs, antimetabolites, topoisomerase 1 and 2 inhibitors, platinum drugs, anti-microtubule agents, and hormones, or derivatives thereof, a molecularly targeted agent, cell cycle signaling agent, modulators of tumor microenvironment, an antibody drug conjugate (ADC), radiation, an immunosuppressant, an enhancer of antigen presenting functions, an enhancer of T/macrophage effector, a drug that influences the purine metabolism, an agonist or antagonist of adenosine receptors, a proteasome inhibitor, a HDAC inhibitor, a TRAIL-R agonist, a chimeric antigen receptor t-cell therapy (CAR-T), an antitumor vaccine, or an immunomodulatory agent targeting PD-1, PD-L1, NKG2A, KIR, CTLA-4, LAG-3, TIM-3, BTLA, VISTA, PD-1H, TIGIT, CD96, STAT3, Arginase-1, HIF-1a, VEGF, CCL2, IDO, Tie2, CSF1, IL-10, IL-13, or IL-23. In some embodiments, the anticancer drug used in combination for the method of the invention can be an immunomodulatory agent targeting CTLA-4, PD-1, or PD-L1.

In the method of the invention, the compound is administered systemically, topically, intratumorally, intrathecally, cutaneously, interstitially, intradermally, intraperidermally, intralesionally, intravesically, or transdermally. In some embodiments, the compound is administered orally, intravenously, intraperitoneally, or intramuscularly. In other embodiments, the compound is administered in the form of cream or gel. In certain embodiments, the compound is administered by injecting to the tumor.

In some embodiments, the route of administration of neoantigen producing drugs are systemic (oral, intravenous, intraperitoneal, intramuscular, etc), topical (cream, gel, and other formulations), intratumoral (injecting to the tumor), intrathecal, cutaneous, interstitial, intradermal, intraperidermal, intralesional, intravesical, or transdermal etc.

In other embodiments, the neoantigen producing drugs are dosed intratumorally. These drugs are dosed intratumorally in various forms; hapten (protein reactive form), prohapten (requiring intracellular chemical transformation), prodrugs of hapten or prohapten (requiring multiple intracellular transformation).

In some embodiments, the neoantigen producing drugs are dosed intratumorally in prohapten form. The prohapten molecules include chemical entities that require one or more chemical transformation to generate reactive hapten molecules. The chemical transformations include but not limit to: oxidation, enzymatic hydrolysis, pH-mediated hydrolysis, glutathione-mediated disulfide bond cleavage. The prohapten molecules are designed to have a broad range of membrane permeability. Cell membrane permeability of prohapten has great impact on the formation of endogenous antigens and/or exogenous antigens and therefore immunogenic responses produced can be adjusted to antigen presentation mediated by MHC Class I or MHC Class II.

In some embodiments, these neoantigen producing molecules are dosed intratumorally in various formulations. The formulations are intended for quick or slow release of the drug from the formulation to the tissue. Those are, but not limited to; aqueous formulations, oleaginous solutions, suspensions or colloids in aqueous and oleaginous solvents, solutions/suspensions/colloids in organic medium using solvents such as, but not limited to; DMSO, ethylene glycol, polyethylene glycol, propylene glycol, oil-in-water emulsions, water-in-oil emulsions, salt forms, larger particles in oil phase of oil-in-water emulsion etc. In certain embodiments, the formulation acts as a depot of the drug in the tumor tissue for the intended release of the drug over several days.

In certain embodiments, the formulations also include water and soluble excipients/co-solvents including, but not limited to, dimethylacetamide (DMA), dimethyl sulfoxide (DMSO), ethanol, glycerin, N-methyl-2-pyrrolidone (NMP), PEG 300, PEG 400, Poloxamer 407, Propylene glycol, Hydroxypropyl-β-cyclodextrin, sulfobutylether-β-cyclodextrin, (Captisol®), α-cyclodextrin, Phospholipids such as hydrogenated soy phosphatidylcholine (HSPC), distearoylphosphatidylglycerol (DSPG), L-α-dimyristoylphosphatidylcholine (DMPC), L-α-dimyristoylphosphatidylglycerol (DMPG). Water insoluble co-solvents include; but are not limited to, beeswax, oleic acid, soy fatty acids, d-α-tocopherol (Vitamin E), Corn oil mono-di-tridiglycerides, medium chain (C8/C10) mono- and diglycerides, long-chain triglycerides such as castor oil, corn oil, cottonseed oil, olive oil, peanut oil, peppermint oil, safflower oil, sesame oil, soybean oil, hydrogenated soybean oil, hydrogenated vegetable oils, medium-chain triglycerides such as caprylic/capric triglycerides, derived from coconut oil or palm seed oil. Surfactants include, but are not limited to, polyoxyl 35 castor oil (cremophor EL), polyoxyl 40 hydrogenated castor oil (cremophor RH 40), polyoxyl 60 hydrogenated castor oil (cremophor RH 60), polysorbate 20 (Tween 20), polysorbate 80 (Tween 80), d-α-tocopheryl polyethylene glycol 1000 succinate (TPGS), solutol HS-15, sorbitan monooleate (Span 20), PEG 300 caprylic/capric glycerides (Softigen 767), PEG 400 caprylic/capric glycerides (Labrasol), PEG 300 oleic glycerides (Labrafil M-1944CS), PEG 300 linoleic glycerides (Labrafil M-2125CS), polyoxyl 8 stearate (PEG 400 monosterate), and polyoxyl 40 stearate (PEG 1750 monosterate).

It will be appreciated by persons skilled in the art that the present invention is not limited by what has been particularly shown and described herein above. Rather the scope of the present invention includes both combinations and subcombinations of the various features described hereinabove as well as variations and modifications which would occur to persons skilled in the art upon reading the specification and which are not in the prior art.

In order that the invention disclosed herein may be more efficiently understood, examples are provided below. It should be understood that these examples are for illustrative purposes only and are not to be construed as limiting the invention in any manner.

### EXAMPLES

### Example 1: Synthesis of N-(2-sulfanylethyl)propenamide (Compound 3)

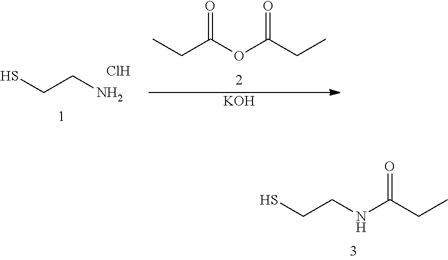

To an ice-cold solution of 2-aminoethane-1-thiol hydrochloride 1 (10.0 g, 83.68 mmol) in H₂O (150 mL) was added aqueous solution of KOH (16.43 g, 292.88 mmol) until the pH 8 is reached followed by addition of propionic anhydride (34.3 g, 251.08 mmol). The resulting reaction mixture was stirred at 0° C. for 1 hour. After the completion of the reaction (TLC monitoring), the reaction mass was basified by addition of solid KOH until pH˜10 and extracted with diethyl ether (100 mL×2 times) for removal of organic impurities. The aqueous part was neutralized by adding 6N HCl, stirred at RT for 1 hour and extracted with dichloromethane (3×100 mL). The combined organics was washed with water (3 times) and brine respectively. The organic part was dried over anhydrous Na₂SO₄, filtered and concentrated under reduced pressure to get desired product 3 as colorless liquid (7.0 g, Yield: 60%).

¹H-NMR (400 MHz, DMSO-d₆): δ 7.89 (br s, 1H), 3.16 (q, J=6.8 Hz, 2H), 2.47 (s, 1H), 2.17-2.21 (m, 2H), 2.03-2.07 (m, 2H) and 0.98 (t, J=7.2 Hz, 3H). LCMS: 134.21 (M+H)⁺, 90.98%.

### Example 2: Synthesis of N-[2-(3,4-dihydroxyphenyl)sulfanylethyl]propenamide (Compound 10)

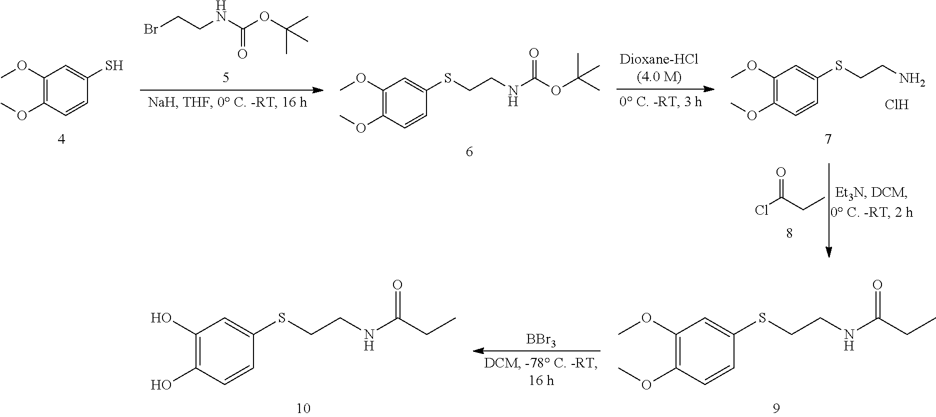

### Example 2a: Preparation of tert-butyl (2-((3,4-dimethoxyphenyl)thio)ethyl)carbamate (6)

To an ice-cold solution of 3,4-dimethoxybenzenethiol 4 (2.5 g, 14.68 mmol) in THF (20 mL) was added sodium hydride (60% dispersion in mineral oil, 0.880 g, 22.02 mmol) portion-wise. The reaction mixture was stirred at 0° C. for 10 min, followed by addition of tert-butyl (2-bromoethyl)carbamate 5 (3.94 g, 17.61 mmol). The resulting reaction mass was stirred at room temperature for 16 h. After the completion of the reaction, the reaction mixture was diluted with water (100 mL) and extracted with EtOAc (3×100 mL). The combined organics was washed with water (3 times) and brine respectively. The organic fraction was dried over anhydrous Na₂SO₄, filtered and concentrated under reduced pressure. The crude was purified over silica gel (100-200M), elution with 10% EtOAc/Hex to get desired product 6 as off white solid (2.7 g, Yield: 58%).

¹H-NMR (400 MHz, CDCl₃): δ 7.05 (d, J=2.0 Hz, 1H), 6.95 (dd, J=2.0 Hz, and J=8.8 Hz, 1H), 6.79 (d, J=8.4 Hz, 1H), 4.88 (br s, 1H), 3.88 (s, 3H), 3.86 (s, 3H), 3.29-3.30 (m, 2H), 2.93 (t, J=6.4 Hz, 2H) and 1.43 (s, 9H). LCMS: 312.16 (M−H)⁺.

### Example 2b: Preparation of 2-((3,4-dimethoxyphenyl)thio)ethan-1-amine hydrochloride (7)

An ice-cold solution of tert-butyl (2-((3,4-dimethoxyphenyl)thio)ethyl)carbamate 6 (2.7 g, 8.61 mmol) in 1,4-dioxane-HCl (4.0 M) was stirred at 0° C. for 15 min, then warmed up to room temperature and stirred for 3 h at same temperature. After the completion of the reaction, the reaction mixture concentrated under reduced pressure to get desired product 7 as off white solid (2.0 g, Yield: Quantitative).

¹H-NMR (400 MHz, DMSO-d₆): δ 8.02 (br s, 3H), 7.01-7.04 (m, 2H), 6.93 (d, J=8.0 Hz, 1H), 3.77 (s, 3H), 3.74 (s, 3H), 3.06-3.09 (m, 2H) and 2.87-2.88 (m, 2H). LCMS: 212.11 (M−H)⁺.

### Example 2c: Preparation of N-(2-((3,4-dimethoxyphenyl)thio)ethyl)propionamide (9)

To an ice-cold solution of 2-((3,4-dimethoxyphenyl)thio)ethan-1-amine hydrochloride 7 (2.0 g, 9.37 mmol) in dichloromethane (25 mL) was added triethylamine (2.6 mL, 18.75 mmol) and propionyl chloride 8 (1.22 mL, 14.05 mmol). The resulting reaction mass was heated at room temperature for 2 h. After the completion of the reaction, the reaction mixture was diluted with water (100 mL) and extracted with EtOAc (3×100 mL). The combined organics was washed with water (3 times) and brine respectively. The organic fraction was dried over anhydrous Na₂SO₄, filtered and concentrated under reduced pressure. The crude was purified over silica gel (100-200M), elution with 50% EtOAc/Hex to get desired product 9 as off white solid (2.0 g, Yield: 79.36%).

¹H-NMR (400 MHz, CDCl₃): δ 7.05 (dd, J=2.0 Hz and 8.4 Hz, 1H), 6.95 (d, J=2.0 Hz, 1H), 6.79 (d, J=8.0 Hz, 1H), 5.79 (br s, 1H), 3.88 (s, 3H), 3.86 (s, 3H), 3.41-3.46 (m, 2H), 2.96 (t, J=6.4 Hz, 2H), 2.14-2.20 (m, 2H) and 1.10 (t, J=7.6 Hz, 3H); LCMS: 270.09 (M+H)⁺, 98.59%.

### Example 2d: Preparation of N-[2-(3,4-dihydroxyphenyl)sulfanylethyl]propenamide (10)

A solution of N-(2-((3,4-dimethoxyphenyl)thio)ethyl)propionamide 9 (2.0 g, 7.43 mmol) in dichloromethane (30 mL) was cooled to −78° C. and followed by slow addition of BBr₃ (1.76 mL, 18.58 mmol). The resulting reaction mixture stirred at room temperature for 16 h. After the completion of the reaction, the reaction mixture was cooled to 0° C. and quenched with methanol (˜4 mL). The reaction mixture was further diluted with water (100 mL) and extracted with ethylacetate (3×100 mL). The combined organics was washed with water (3 times) and brine respectively. The organic fraction was dried over anhydrous Na₂SO₄, filtered and concentrated under reduced pressure. The crude was purified over combiflash, elution with 70% EtOAc/Hex to get desired product 10 as viscous liquid (1.0 g, Yield: 55.8%).

¹H-NMR (400 MHz, MeOD): δ 6.89 (d, J=2.0 Hz, 1H), 6.79 (dd, J=2.0 Hz and 8.0 Hz, 1H), 6.69 (d, J=8.0 Hz, 1H), 3.28-3.34 (m, 2H), 2.86 (t, J=6.8 Hz, 2H), 2.16 (q, J=7.6 Hz, 2H) and 1.07 (t, J=7.6 Hz, 3H). LCMS: 242.01 (M+H)⁺, 99.79%.

### Example 3: Synthesis of N-[2-(2,5-dihydroxyphenyl)sulfanylethyl]propenamide (Compound 12)

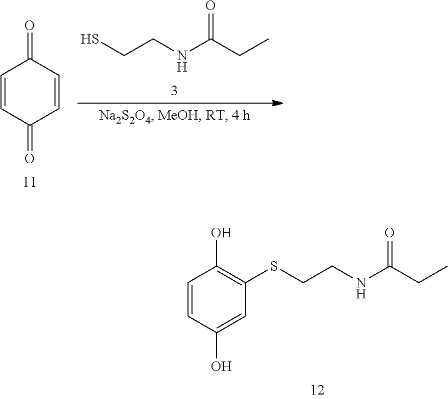

To an ice-cold solution of 1,4-benzoquinone 11 (1.0 g, 9.25 mmol) in methanol (20 mL) was added N-(2-sulfanylethyl)propenamide 3 (1.47 g, 11.1 mmol) and sodium dithionite (3.22 g, 18.5 mmol). The resulting reaction mixture stirred at room temperature for 16 h. After the completion of the reaction, the reaction mixture was cooled to 0° C. diluted with water (100 mL) and extracted with ethylacetate (3×100 mL). The combined organics was washed with water (3 times) and brine respectively. The organic fraction was dried over anhydrous Na₂SO₄, filtered and concentrated under reduced pressure. The crude was purified over combiflash, elution with 70% EtOAc/Hex to get desired product 12 as viscous liquid (1.05 g, Yield: 56%).

¹H-NMR (400 MHz, MeOD): δ 6.83 (d, J=2.8 Hz, 1H), 6.66 (d, J=8.8 Hz, 1H), 6.56 (dd, J=2.8 Hz and 8.8 Hz, 1H), 3.31-3.34 (m, 2H), 2.90 (t, J=6.8 Hz, 2H), 2.14 (q, J=7.6 Hz, 2H) and 1.08 (t, J=7.6 Hz, 3H). LCMS: 242.04 (M+H)⁺, 97.91%.

### Example 4: Synthesis of N-[2-(2,3-dihydroxyphenyl)sulfanylethyl]propenamide (Compound 14)

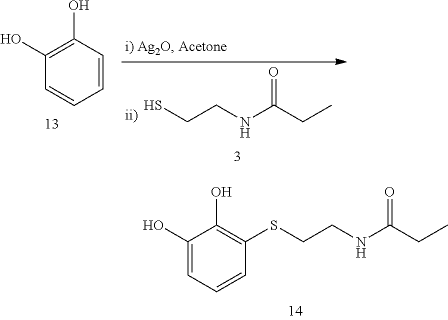

To an ice-cold solution of catechol 13 (5.0 g, 45.45 mmol) in acetone (100 mL) was added silver oxide (21.0 g, 90.90 mm). The reaction mixture was stirred at 0° C. for 2 h. After the completion of the reaction, the reaction mixture was filtered through celite bed and filtrate was concentrated under reduced pressure to get the crude product, which was used for further next step without purification. To an ice cold solution of crude o-benzoquinone (obtained from catechol 13) in dichloromethane (100 mL) was added pyridine (7.34 mL, 90.90 mmol) and N-(2-sulfanylethyl)propenamide 3 (7.25 g, 54.54 mmol). The resulting reaction mass stirred at room temperature for 16 h. After the completion of the reaction, the reaction mass was cooled to 0° C., adjust pH˜6-7 using iN HCl and extracted with ethylacetate (3×100 mL). The combined organics was washed with water (3 times) and brine respectively. The organic fraction was dried over anhydrous Na₂SO₄, filtered and concentrated under reduced pressure. The crude was purified over combiflash, elution with 70% EtOAc/Hex to get desired product 14 as viscous liquid (900 mg, Yield: 8.2%).

¹H-NMR (400 MHz, MeOD): δ 6.87 (dd, J=1.6 Hz and 8.0 Hz, 1H), 6.72 (dd, J=1.6 Hz and 8.0 Hz, 1H), 6.60-6.63 (m, 1H), 3.29-3.34 (m, 2H), 2.87 (t, J=6.8 Hz, 2H), 2.13 (q, J=7.6 Hz, 2H) and 1.07 (t, J=7.6 Hz, 3H). LCMS: 242.08 (M+H)⁺, 99.84%.

### Example 5: Synthesis of 4-(2-aminoethylsulfanyl)benzene-1,2-diol (Compound 15)

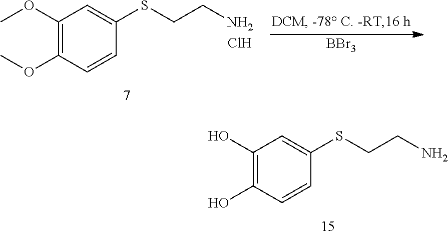

A solution of 2-((3,4-dimethoxyphenyl)thio)ethan-1-amine hydrochloride 7 (1.0 eq) in dichloromethane was cooled to −78° C. and followed by slow addition of BBr₃ (3.0 eq). The resulting reaction mixture was stirred at room temperature for 16 h. After the completion of the reaction, the reaction mixture was cooled to 0° C. and quenched with methanol (˜4 mL). The reaction mixture was diluted with water (100 mL) and extracted with ethylacetate (3×100 mL). The combined organics was washed with water (3 times) and brine respectively. The organic fraction was dried over anhydrous Na₂SO₄, filtered and concentrated under reduced pressure. The crude was purified over prep-HPLC to get desired product 15 as viscous liquid.

¹H-NMR (400 MHz, MeOD): δ 6.94 (d, J=2.0 Hz, 1H), 6.86 (dd, J=2.0 Hz and 8.0 Hz, 1H), 6.74 (d, J=8.0 Hz, 1H) and 3.00 (s, 4H). LC-MS: 184.01 (M−H)⁺.

### Example 5a: General Method A: Amide Synthesis from Compound 7

To an ice-cold solution of 2-((3,4-dimethoxyphenyl)thio)ethan-1-amine hydrochloride 7 (1.0 eq) in dichloromethane was added triethylamine (2.0 eq) and respective acid chlorides (1.2 eq). The resulting reaction mixture was stirred at room temperature for 2 h. After the completion of the reaction, the reaction mixture was diluted with water (100 mL) and extracted with ethylacetate (3×100 mL). The combined organics was washed with water (3 times) and brine respectively. The organic fraction was dried over anhydrous Na₂SO₄, filtered and concentrated under reduced pressure. The crude was purified over silica gel (100-200M), elution with 50% ethylacetate/hexane to get desired products.

### Example 6: Synthesis of N-[2-(3,4-dihydroxyphenyl)sulfanylethyl]heptanamide (Compound 18)

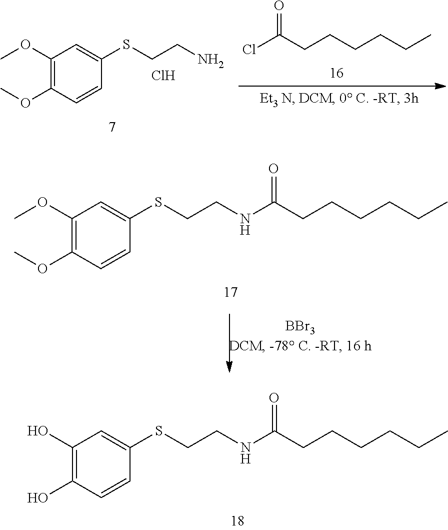

### Example 6a: Synthesis of N-[2-(3,4-dimethoxyphenyl)sulfanylethyl]heptanamide (Compound 17)

This compound was synthesized according to the Method A mentioned above. This product was isolated as an off-white solid.

¹H-NMR (400 MHz, CDCl₃): δ 6.99 (d, J=2.0 Hz, 1H), 6.96 (s, 1H), 6.80 (d, J=8.4 Hz, 1H), 5.77 (br s, 1H), 3.88 (s, 3H), 3.86 (s, 3H), 3.41-3.45 (m, 2H), 2.96 (t, J=6.0 Hz, 2H), 2.11 (m, 2H), 1.25-1.28 (m, 8H) and 0.86 (t, J=6.8 Hz, 3H). MS: 326.17 (M+H)⁺.

### Example 6b: Synthesis of N-[2-(3,4-dihydroxyphenyl)sulfanylethyl]heptanamide (Compound 18)

The conversion of compound 17 to compound 18 was done using the protocol described for compound 15.

¹H-NMR (400 MHz, MeOD): δ 6.88 (d, J=2.0 Hz, 1H), 6.78 (dd, J=2.0 Hz and 8.0 Hz, 1H), 6.69 (d, J=8.4 Hz, 1H), 3.28-3.31 (m, 2H), 2.84 (t, J=6.8 Hz, 2H), 2.12 (q, J=6.8 Hz, 2H), 1.55-1.58 (m, 2H), 1.25-1.33 (m, 6H) and 0.88 (t, J=7.2 Hz, 3H). LC-MS: 298.20 (M+H)⁺, 99.46%.

### Example 7: Synthesis of N-[2-(3,4-dihydroxyphenyl)sulfanylethyl]undecanamide (Compound 21)

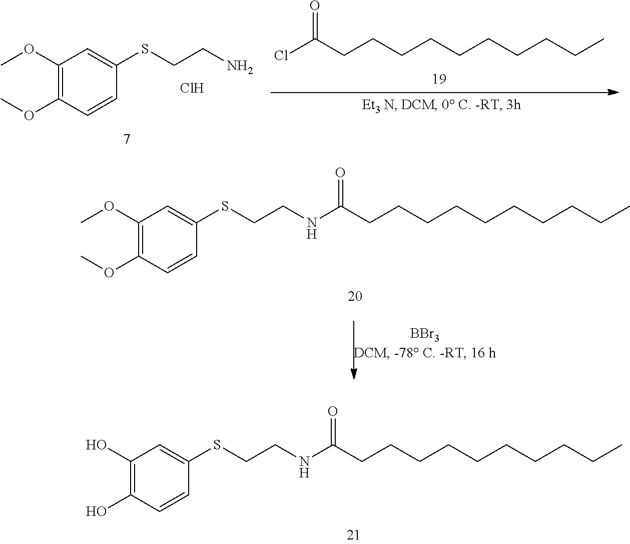 [SMILES: COc1ccc(SCCN)cc1OC ; Cl ; CCCCCCCCCCC(=O)Cl ; CCCCCCCCCCC(=O)NCCSc1ccc(OC)c(OC)c1 ; CCCCCCCCCCC(=O)NCCSc1ccc(O)c(O)c1]

### Example 7a: Synthesis of N-[2-(3,4-dimethoxyphenyl)sulfanylethyl]undecanamide (Compound 20)

The compound 20 was synthesized using the Method A mentioned above. Product 20 was isolated as a solid powder.

¹H-NMR (400 MHz, CDCl₃): δ 6.98 (d, J=2.0 Hz, 1H), 6.97 (s, 1H), 6.81 (d, J=8.4 Hz, 1H), 5.78 (br s, 1H), 3.88 (s, 3H), 3.86 (s, 3H), 3.42-3.45 (m, 2H), 2.97 (t, J=6.0 Hz, 2H), 2.12 (m, 2H), 1.25-1.28 (m, 16H) and 0.87 (t, J=6.8 Hz, 3H). LC-MS: 382.20 (M+H)⁺.

### Example 7b: Synthesis of N-[2-(3,4-dihydroxyphenyl)sulfanylethyl]undecanamide (Compound 21)

Compound 21 was obtained from compound 20 using the general method described for compound 15.

¹H-NMR (400 MHz, MeOD): δ 6.88 (d, J=2.0 Hz, 1H), 6.78 (dd, J=2.0 Hz and 8.0 Hz, 1H), 6.69 (d, J=8.0 Hz, 1H), 3.28-3.34 (m, 2H), 2.84 (t, J=6.8 Hz, 2H), 2.12 (q, J=6.8 Hz, 2H), 1.55-1.58 (m, 2H), 1.25-1.29 (m, 14H) and 0.87 (t, J=7.2 Hz, 3H). LC-MS: 354.42 (M+H)⁺,95.88%.

### Example 8: Synthesis of N-[2-(3,4-dihydroxyphenyl)sulfanylethyl]pentanamide (Compound 24)

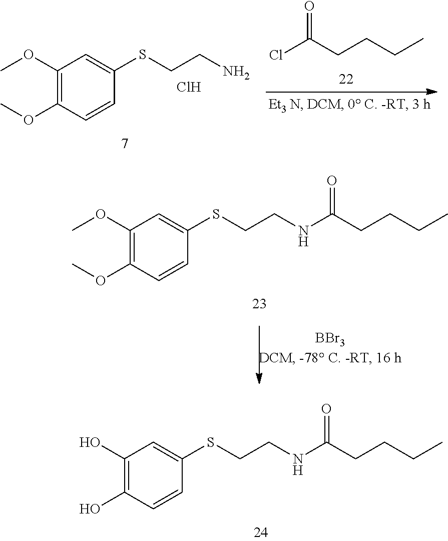

### Example 8a: Synthesis of N-[2-(3,4-dimethoxyphenyl)sulfanylethyl]undecanamide (Compound 23)

The compound 23 was synthesized from compound 5 using the Method A descried above.

¹H-NMR (400 MHz, CDCl₃): δ 6.99 (d, J=2.0 Hz, 1H), 6.96 (s, 1H), 6.80 (d, J=8.4 Hz, 1H), 5.77 (br s, 1H), 3.88 (s, 3H), 3.86 (s, 3H), 3.41-3.45 (m, 2H), 2.96 (t, J=6.0 Hz, 2H), 2.11 (m, 2H), 1.25-1.28 (m, 4H) and 0.86 (t, J=6.8 Hz, 3H). LC-MS: 298.10 (M+H)⁺.

### Example 8b: Synthesis of N-[2-(3,4-dihydroxyphenyl)sulfanylethyl]pentanamide (Compound 24)

The compound 24 was obtained from compound 23 using the demethylation protocol described for compound 15.

¹H-NMR (400 MHz, MeOD): δ. 6.88 (d, J=2.1 Hz, 1H), 6.80 (dd, J=2.1 and 8.2 Hz, 1H), 6.7 (d, J=8.1 Hz, 1H), 2.86 (t, J=7.2 Hz, 2H), 2.14 (t, J=7.4 Hz, 2H), 1.55 (pent, J=7.36 Hz, 2H), 1.38-1.28 (m, 4H), 0.92 (t, J=7.3 Hz, 3H); LCMS: 270.14 (M+H)⁺.

### Example 9: Synthesis of N-[2-(3,4-dihydroxyphenyl)sulfanylethyl]cyclopentanecarboxamide (Compound 27)

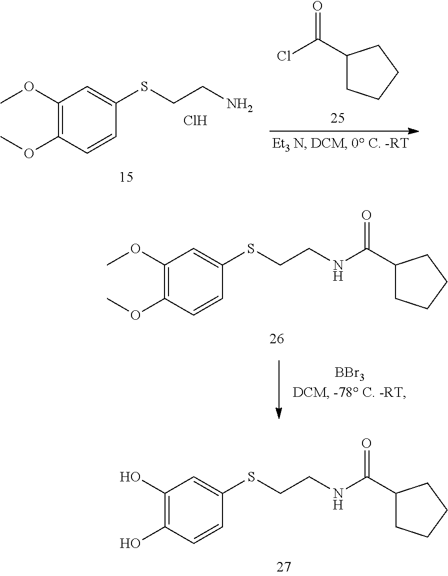 [SMILES: COc1ccc(SCCN)cc1OC ; Cl ; O=C(Cl)C1CCCC1 ; COc1ccc(SCCNC(=O)C2CCCC2)cc1OC ; O=C(NCCSc1ccc(O)c(O)c1)C1CCCC1]

Compound 27 was synthesized using similar protocol described for compound 18. During the isolation, some oxidation to the corresponding was observed (based on LC-MS). LC-MS: 282.32 (M+1).

### Example 10: Synthesis of 3-(5,5-difluoro-1,3-dimethyl-dipyrrolo[3,1-c:2′,1′-g][1,3,2]diazaborinin-4-ium-7-yl)-N-[2-(3,4-dihydroxyphenyl)sulfanylethyl]propenamide (Compound 29)

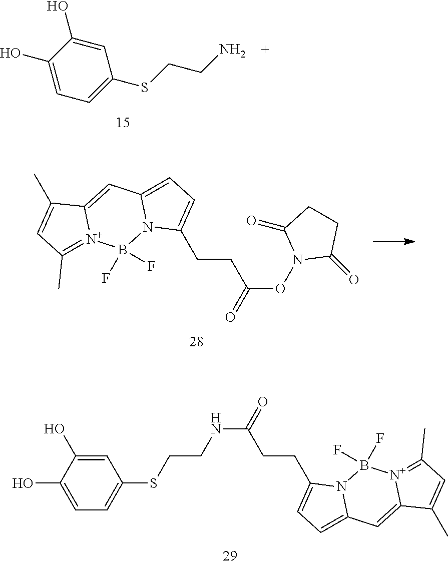

To a solution of BODIPY-FL (28, 100 mg) and amine 15 (50 mg) in 5 mL tetrahydrofuran was added 0.2 mL of triethylamine and the mixture stirred overnight. LC/MS indicated presence of BODIPY-FL. Additional 20 mg of amine 15 was added and stirred for 3 h; Solvent was removed by rotavapor and crude product was purified by chromatography (dichloromethane:methanol—98:2)). Pure fractions were pooled together and concentrated to get target compound 29 as red color powder. LCMS: 460.24 (M+H)⁺.

### Example 11: Synthesis of 3-pentadecylbenzene-1,2-diol (Compound 34)

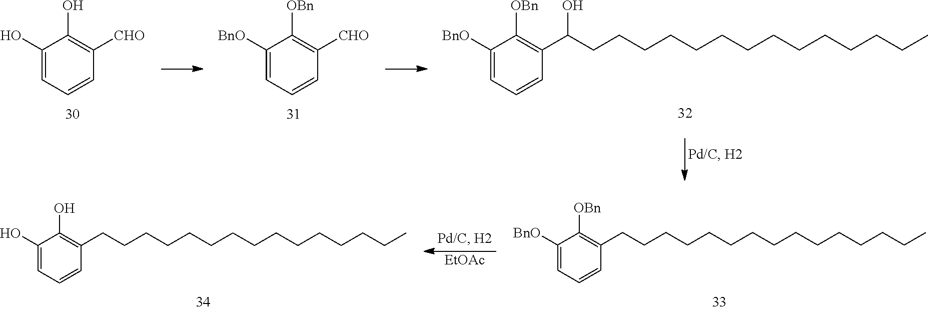 [SMILES: "EtOAc" ; O=Cc1cccc(O)c1O ; O=Cc1cccc(OCc2ccccc2)c1OCc1ccccc1 ; CCCCCCCCCCCCCCC(O)c1cccc(OCc2ccccc2)c1OCc1ccccc1 ; CCCCCCCCCCCCCCCc1cccc(O)c1O ; CCCCCCCCCCCCCCCc1cccc(OCc2ccccc2)c1OCc1ccccc1]

The compound 34 was synthesized using the protocol described in the patent WO 2009/146131 A2, however slightly modified. For the conversion of compound 32 to compound 34, double hydrogenation steps were performed that led to the isolation of the intermediate 33. Compound 34 was obtained as off-white powder and analytical parameters (NMR and LCMS) agree with the description made in the above-mentioned patent.

### Example 12: Synthesis of 3-pentadecyl-1,2-benzoquinone (Compound S2)

Compound S2 is synthesized from 3-pentadecylbenzene-1,2-diol using oxidation. The oxidation is achieved using several conditions using enzymes such as myeloperoxidases, CYP enzymes, organic and inorganic oxidants such as hydrogen peroxide, Pb(OAc)₄, O₂ etc.

In a typical experimental procedure, compound S1 is oxidized with Pb(OAc)4 in dichloromethane to afford compound S2 using the protocol from the literature (Knapp et al., Journal of Organic Chemistry, 1985, 50, 4996-4998)

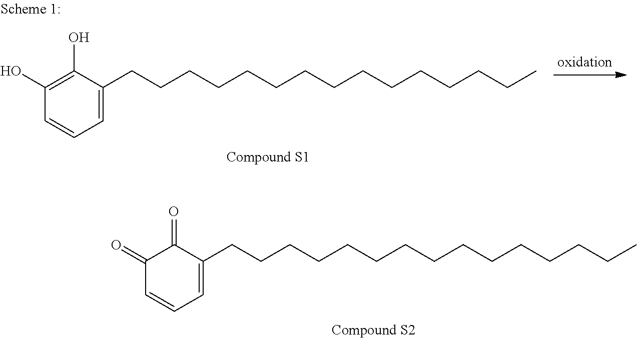 [SMILES: CCCCCCCCCCCCCCCc1cccc(O)c1O ; CCCCCCCCCCCCCCCC1=CC=CC(=O)C1=O]

### Example 13: Synthesis of 3-[(Z)-pentadec-10-enyl]benzene-1,2-diol (Compound 371)

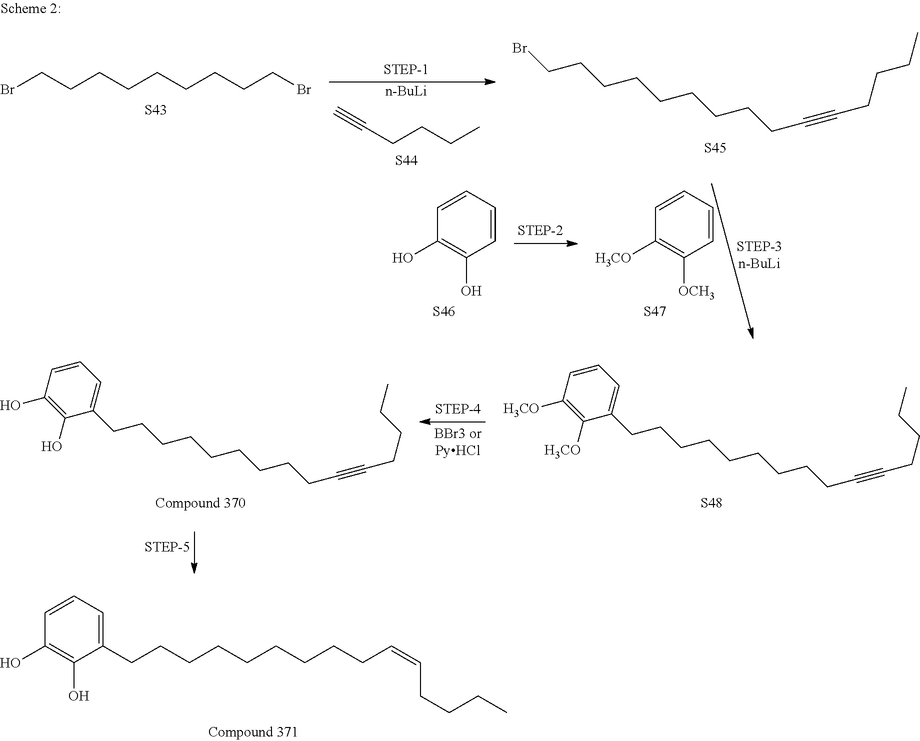

### Example 13a: Synthesis of 15-bromopentadec-5-yne S45

To a stirred solution of hex-1-yne in anhydrous THF at −78° C. was added a solution of 1.6M n-BuLi dropwise. The mixture was slowly brought to 0° C. and stirred for 30 min. The mixture was cooled again to −78° C. and 1,9-dibromononane in THF was added dropwise. After the completion of the addition, the mixture was stirred for 4 h and quenched with a saturated solution of NH₄Cl. After the separation of the layers, the aqueous layer was extracted with ethyl acetate. The organics were dried over anhydrous sodium sulfate and concentrated to obtain crude product that was used for the next step.

### Example 13b: Synthesis of 1,2-dimethoxy-3-pentadec-10-ynyl-benzene S48

To a stirred solution of o-xylene in THF at −78° C. was added 1.6 M n-BuLi in hexanes dropwise. The mixture was stirred for 30 min and was then added a solution of 15-bromopentadec-5-yne in THF drop wise. The mixture thus obtained was further stirred for 5 h and quenched with a saturated solution of NH₄Cl. After the aqueous work up the organics were dried over anhydrous sodium sulfate and concentrated. A brief column chromatography over silica gel afforded the desired product S48.

### Example 13c: Synthesis of 3-pentadec-10-ynylbenzene-1,2-diol (Compound 370)

To a stirred solution of 1,2-dimethyl-3-pentadec-10-ynyl-benzene in dry chloroform at 0° C., was added a solution of boron tribromide in chloroform dropwise. After the addition the mixture was stirred at 0° C. for 5 h and quenched with ice-water. The mixture was thoroughly washed with water and brine and concentrated. Purification with column chromatography on silica gel afforded compound 362.

### Example 13d: Synthesis of 3-[(Z)-pentadec-10-enyl]benzene-1,2-diol (compound 371)

The compound 370 was hydrogenated in ethyl acetate using 5% Pd—BaSO₄ catalyst and the reaction was monitored for completion. Filtration of the catalyst followed by evaporation of the solvents and brief chromatography on silica afforded compound 363 as thick oil.

LC-MS: Mass calculated for C₂₁H₃₄O₂ is 318.23, observed: 319.1 (M⁺+1).

### Example 14: Synthesis of (4Z)-4-[(7-chloro-4-quinolyl)imino]-2-(diethylaminomethyl)cyclohexa-2,5-dien-1-one (Compound 90)

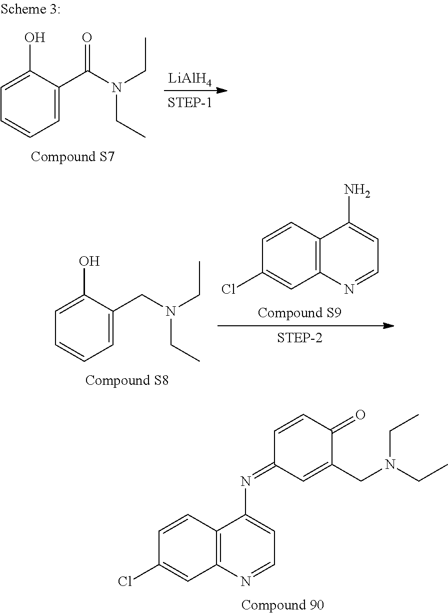

### Example 14a: Synthesis of Compound S8

The compound S8 was synthesized from compound S7 by the lithium aluminum hydride reduction according to literature (Assimomytis et al., Synlett, 2009, 2777-2782).

### Example 14b: Synthesis of (4Z)-4-[(7-chloro-4-quinolyl)imino]-2-(diethylaminomethyl)cyclohexa-2,5-dien-1-one

The oxidative addition of the compound S9 to the phenolic derivative compound S8 is achieved using K₃[Fe(CN)₆], using the protocol reported in the literature (Kubo et al., Journal of Organic Chemistry, 1996, 61, 3758-3765).

### Example 15: IFNγ Assay for Testing the Activity of Compounds of the Invention

Splenocytes from mice were collected and prepared following standard procedure.

Preparation of Melanoma Cells
B16 Melanoma cells were
a. thawed;
b. washed in PBS;
c. re-suspended;
d. irradiated at 2500 cGy;
e. washed again in PBS;
f. re-suspended to 1×10⁷/mL.

Generation of Modified Cells
i. 1 mL “compound” solution (10 mM, pH 7.4) was added to 1 mL cell suspension (note: the term “compound” refers to the compound of the invention);
ii. After shaking, the cells were incubated with “compound” at 37° C. for 30 min;
iii. The reaction was stopped by adding 20 mL medium supplemented with 10% FCS (fetal bovine serum);
iv. The cells were then centrifuged and washed three times with PBS to remove unbound “compound” as well as compound-modified serum proteins;
v. Cells were readjusted to 1×10⁷/mL.

Measuring of T-cell response to compound induced antigens by IFNγ production.
i. Prepared 3-fold dilution of antigens (compound-modified melanoma cells): 10, 5, 2.5×10⁴ cells/mL;
ii. Mouse splenocytes (1×10⁵/well) were stimulated with compound-modified melanoma cells (10, 5, 2.5×10⁴/well) in 200 μL complete medium plus 5% human AB serum in 96-well U-bottom plates. The plates were placed in a humidified 37° C., 5% CO₂ incubator for 72 hours.
iii. The supernatants were collected for IFNγ assay.
iv. As control, mouse splenocytes (1×10⁵/well) were treated with irradiated unmodified melanoma cells (10, 5, 2.5×10⁴/well) in 200 μL complete medium plus 5% human AB serum in 96-well U-bottom plates. The plates were placed in a humidified 37° C., 5% CO₂ incubator for 72 hours. The supernatants were collected for IFNγ assay.

Analysis
a. Supernatants were placed in 96 well polypropylene plates.
b. Analyze IFNγ using ELISA kit or FACS
   i. As controls, analysis were performed with supernatants generated from step 4 with modification of (1) with unmodified melanoma cells and (2) without melanoma cells

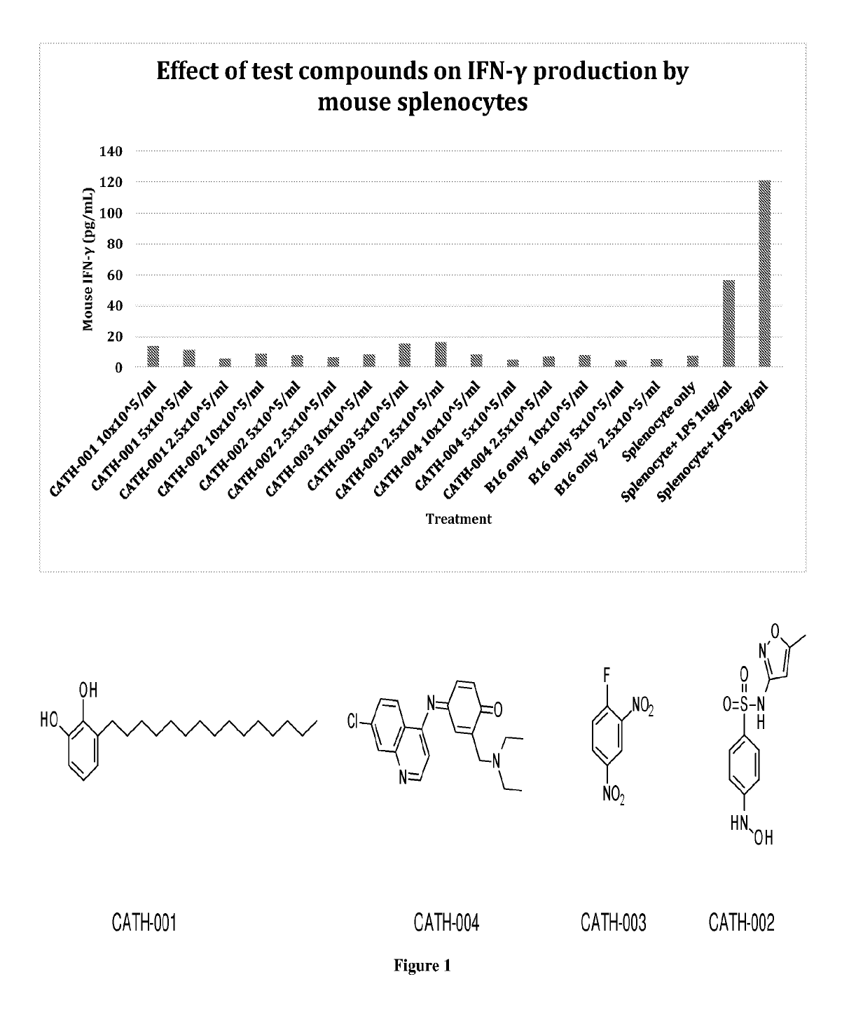

Results are provided in FIG. 1.

### Example 16: Cytotoxicity Assay

Cell lines used: CT26, B16F10 and NIH3T3

Concentration range of drug to be tested: 50 nM to 50 uM

Cells were plated in a 96 well plate and when they are about 50% confluent the drug was added in DMSO so that the final concentration of DMSO is 0.1% (v/v) of less and incubated for 72 hours. The cell viability is then determined using Cell Titer GLO.

TABLE 1

| GC₅₀ (mM) | Compound 10 | Compound 12 | Compound 14 |
|---|---|---|---|
| CT26 Colon Cancer | >50 | 14.2 | 9.9 |
| B16F10 Melanoma | >50 | >50 | 4.7 |
| NIH 3T3 Non-tumor | >50 | 3.3 | >50 |

### Example 17: In Vivo Efficacy Studies

CT26 cells were cultured in RPMI-1640 cell culture medium supplemented with 10% FBS and 1% penicillin-streptomycin. The cells were harvested by trypsinization, when they reached 70-80% confluence. Balb/c female mice were inoculated with these CT26 cells subcutaneously on the dorsal right flank. Prior to inoculation, the skin on the injection site was swabbed with 70% ethanol. Hair around the area of cell inoculation was removed using a standard clipper (Andis AGR+4.8). CT26 cells (P14) in serum free medium (1.0×10⁶/50 μL) were mixed with matrigel at 1:1 and a total volume of 100 μL was injected to each animal with a 1 mL BD syringe attached to a 23^{1/2} gauge needle.

CT26 allografts were measured after 7 days of cell inoculation when the average tumor volume (±SEM) reached approximately 140±10 mm³. Thirty two mice based on tumor volume were randomized into 4 groups (shown in the Table 2) with 8 mice in each group by maintaining the average tumor size as specified above.

TABLE 2

| | | | Dosing | | |
|---|---|---|---|---|---|
| Group | Treatment | Route | frequency | Dose/mouse | n/group |
| 1 | Vehicle | i.t. | QD days | 50 μl | 8 |
| | control | (Intra- | 1-5 | | |
| | (PEG-300) | tumor) | | | |
| 2 | Compound 10 | i.t. | QD days | 1 mg/50 μl | 8 |
| | | (Intra- | 1-5 | | |
| | | tumor) | | | |
| 4 | mouse PD-1 | i.p. | QD days | 0.1 mg | 8 |
| | antibody | | 1, 5 | | |
| 3 | Compound | i.t. and | QD days | Compound 10: | 8 |
| | 10 + | i.p. | 1-5 and | 1 mg mouse | |
| | mouse PD-1 | | QD days | PD-1 antibody: | |
| | antibody | | 1, 5 | 0.1 mg | |

i.t.: intratumoral
i.p.: intraperitoneal

Drug and Vehicle Information

a) Vehicle: PEG300, compound 10 is dosed intratumorally as a solution in PEG300.

b) Tumor growth inhibition (TGI) is calculated is calculated using the following formula:

% TGI=[1−(Treatment TV_{Final}−Treatment TV_{Initial})/(Control TV_{Final}−Control TV_{Initial})]*100

c) Change in body weight (% BW) is calculated using the following formula:

% BW change=(BW_{Final}−BW_{Initial})/(BW_{Initial})*100

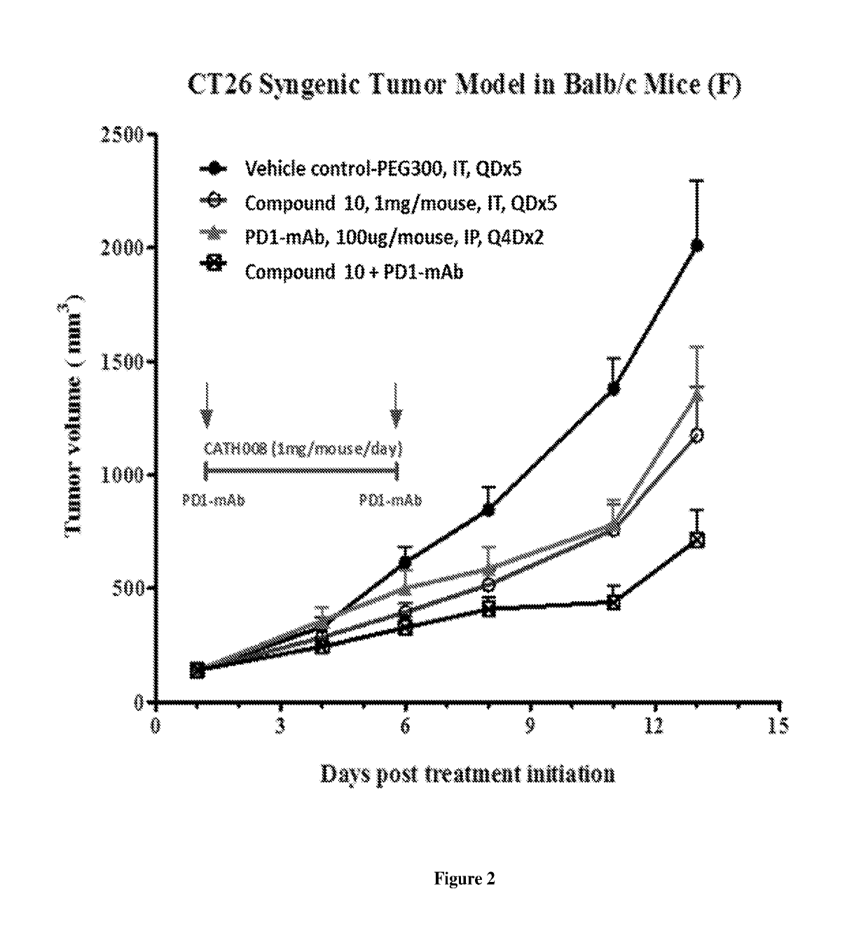

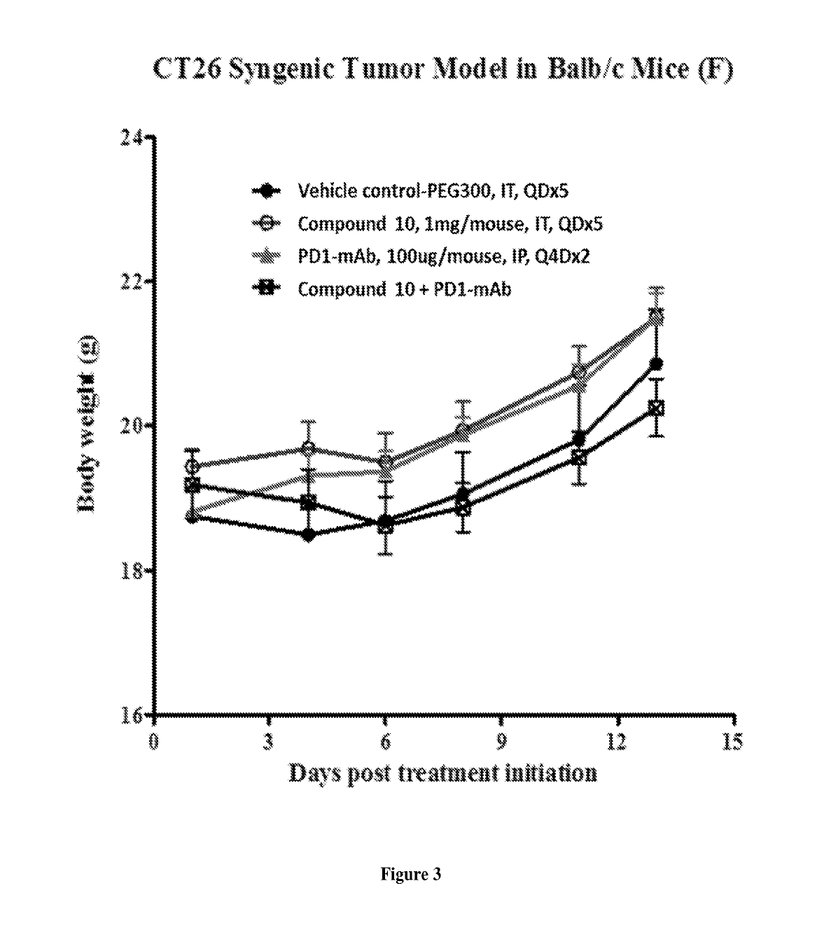

Results are shown in FIGS. 2 and 3.

### Example 18: In Vivo Immune Cell Infiltration Assay

CT26 mouse colon cancer cells were cultured in complete RPMI 1640 (10% FBS, 1% penicillin/streptomycin) in culture treated, canted, vented flasks. When the cells were ˜70% confluent, they were harvested by rinsing with sterile PBS and detached from the flasks with 0.25% Trypsin EDTA solution, washed in C-RPMI, and resuspended in sterile PBS. Viable cell concentration was determined using a hemocytometer and trypan blue staining. Cells were suspended in sterile PBS at a concentration of 2×107 cells/mL. Cell viability before injections was >95%.

Female BALB/c mice that were ˜8-9 weeks old were inoculated with 50 uL of the CT26 cell suspension (1×106 cells) by injecting into the subcutaneous space on the left flank using a 27-gauge needle. Tumors were allowed to develop and when tumors were ˜100 mm³ (average), they were randomized across two groups. The group 1 was treated with vehicle (PEG300) by injecting 50 uL directly into the tumor (intratumoral dose) on day 1 to 5 (qd) while group 2 was treated with compound 10 (1 mg/mouse/day) as a solution in PEG300 (50 uL) on day 1 to 5 (qd). Clinical observations including the body weight change and tumor volumes were recorded.

Tumors were harvested on day 14 (from the first dose) and weighed. Tumors were then minced with a sterile scalpel to pieces <1 mm in diameter, homogenized against a 70 um nylon filter, and digested in HBSS containing: collagenase at 28 units/mil and DNaseI at 2 mg/ml for 45 minutes at 37° C. The tumor single cell suspensions were washed, filtered, prepped and stained for multicolor FACS analysis on a MACSQuant flow cytometer using the two marker panels; namely CD8 cells and CD4 cells. Cell suspensions were stained with fluorochrome conjugated antibodies and a dead cell marker and examined with flow cytometry. CD4 T-cells, CD8−T cells, and total CD3+ cells are displayed for each group. Values are expressed as a frequency (percentage of total infiltrating CD45+ cells)+SEM.

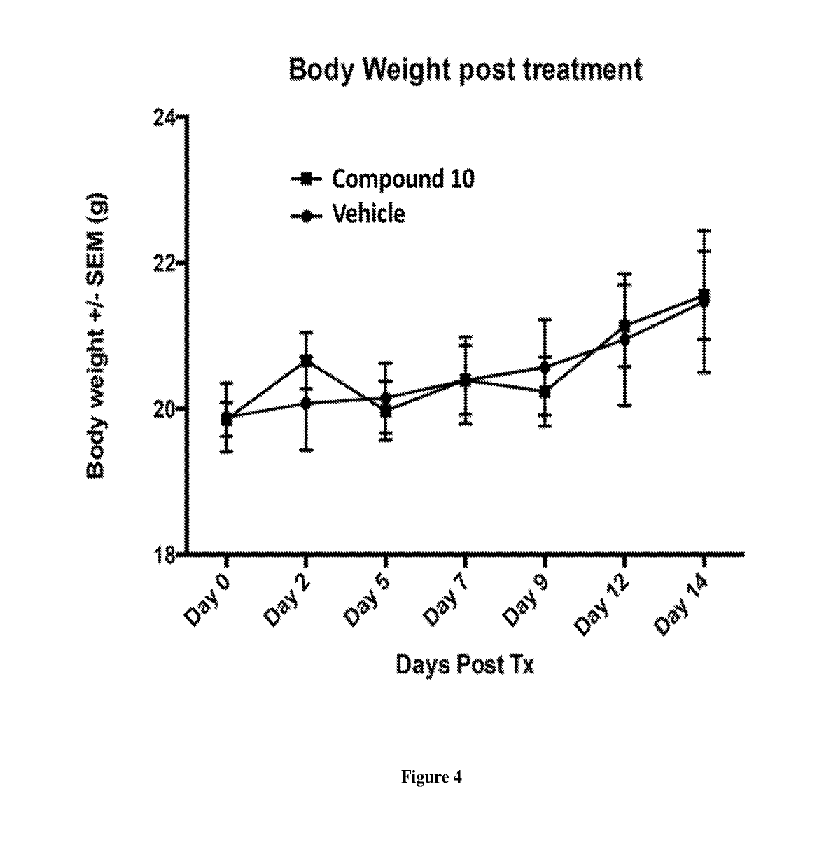

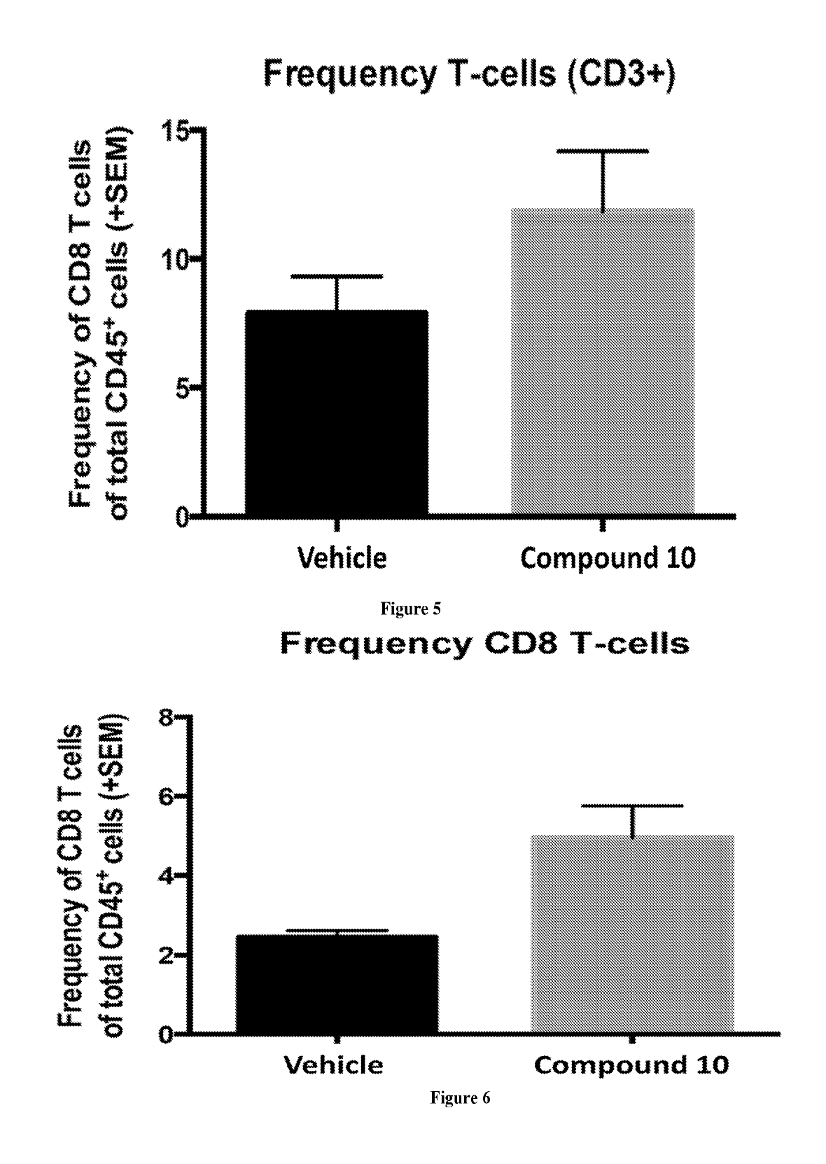

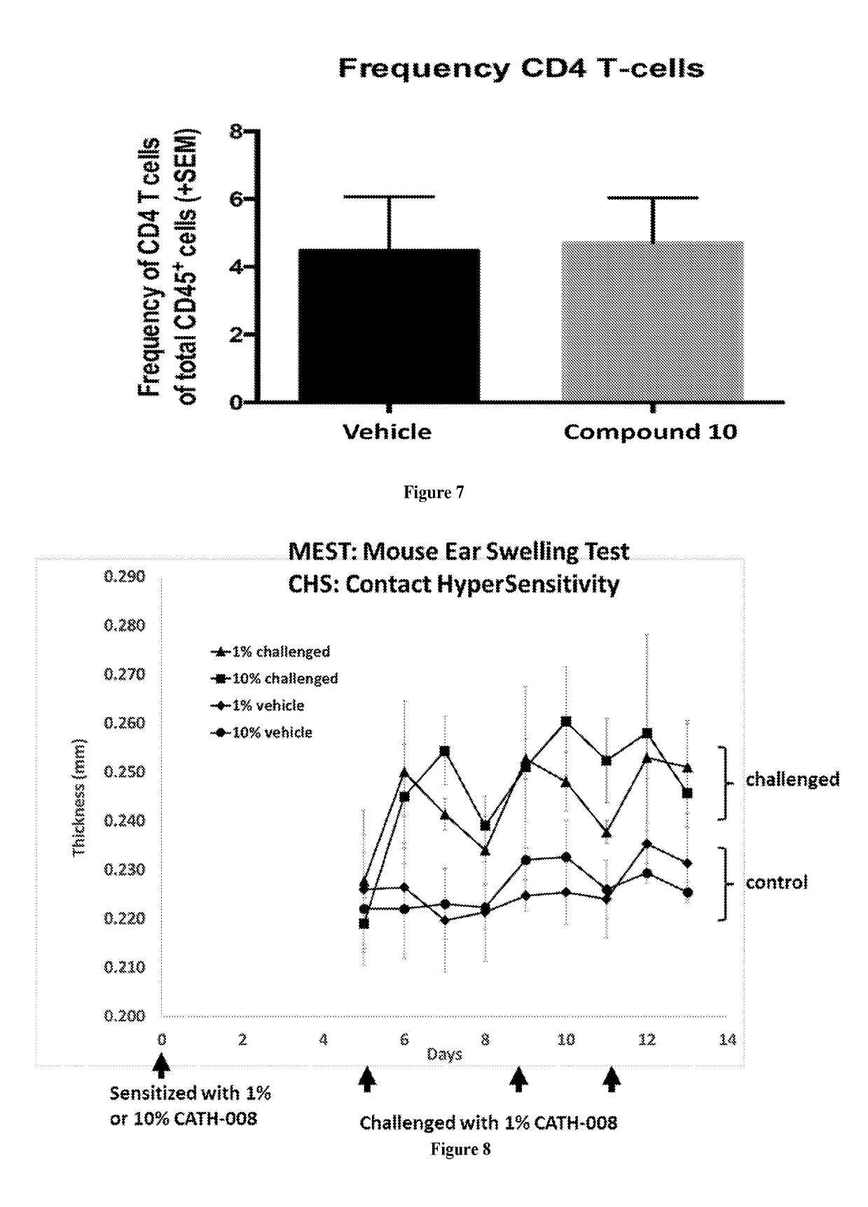

The results are provided in FIGS. 4-7.

### Example 19: Mouse Ear Sensitivity Test

8-week-old C57BL/6 mice were sensitized with a solution (1% or 10% (w/w)) of the drug in 4:1 (v/v) acetone:olive oil mixture (50 uL) on the abdomen (shaven) on day 0. The animals were then challenged by applying a 1% (w/w) solution (25 uL) of the drug in 4:1 (v/v) acetone:olive oil mixture on days 5, 8 and 11 on one year. The other ear was left untreated. Clinical observations were made 24 h and 48 h post challenge. The ear thickness was measured and swelling was calculated by subtracting the pre-challenge value from the post challenge value as shown in FIG. 8.

### Example 20: Covalent Bond Formation of Compound 10 with Cysteine

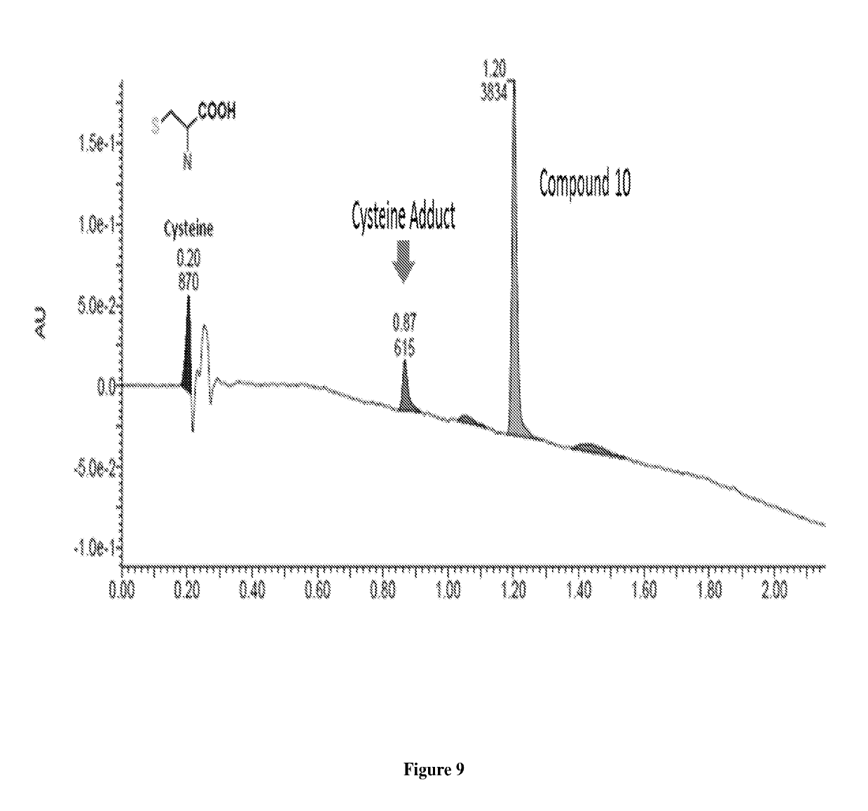

A 1 mg/mL solution of compound 10 in methanol was mixed with a 5 mg/mL solution of cysteine in methanol in equal portions and stirred in open air for 30 min. The resultant mixture was injected into the LC-MS to determine the extent of C—S bond link. The results are provided in FIG. 9.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound, or a pharmaceutically acceptable salt, a conjugate, or a prodrug thereof, wherein said compound is represented by Formula 1 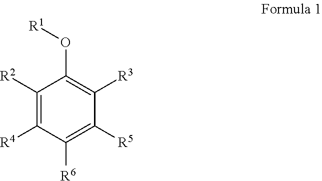
wherein
R¹ is H, PG, or PDG;
R² and R⁴ are each independently H, halogen, alkyl, haloalkyl, or aryl, wherein said alkyl, haloalkyl, and aryl are each optionally substituted with —NR⁷R^{7′}, —OR⁸, —SR^{8′}, or —SO₂—R⁸;
R³ is H, halogen, OH, —O-PG, —O-PDG, alkyl, haloalkyl, cycloalkyl, aryl, heteroaryl, —NR⁹R^{9′}; or R³ and R¹, together with the atoms to which they are attached, form a heterocyclic group;
R⁵ and R⁶ are independently H, halogen, OH, —OPG, —OPDG, alkyl, alkenyl, alkynyl, cycloalkyl, or heterocycloalkyl, wherein said alkyl, alkenyl, alkynyl, cycloalkyl, and heterocycloalkyl are each optionally substituted with NR⁹R^{9′} or OR^{10′}, and wherein when R⁵ is H, R⁶ is not H; and when R⁶ is H, R⁵ is not H; or
R⁵ and R⁶ each are independently selected from the group consisting of —S-alkyl, —S—(CH₂)ₚNR⁹R^{9′}, —S—(CH₂)ₚOR¹⁰, —O-alkyl, —O—(CH₂)ₚNR⁹R^{9′}, —O—(CH₂)ₚOR¹⁰, —NR¹¹R¹², NR¹¹—(CH₂)ₚNR⁹R^{9′}, and —NR¹¹—(CH₂)ₚOR¹⁰;
R⁷, R^{7′}, R⁹, and R^{9′} are each independently H, alkyl, aryl, —CO-alkyl, —CO-cycloalkyl, —CO-aryl, —CO-heteroaryl, —SO₂-alkyl, —SO₂-cycloalkyl, —SO₂-aryl, or —SO₂-heterocycloalkyl, wherein said alkyl, cycloalkyl, heterocycloalkyl, aryl, and heteroaryl are each optionally substituted with a substituent independently selected from halo, amino, C₁₋₃ alkylamino, di-C₁₋₃ alkylamino, NO₂, C₁₋₅ alkyl, O—C₁₋₃ alkyl, cyano, C₁₋₃ haloalkyl, O—C₁₋₃ haloalkyl, COOH, cycloalkyl, heterocycloalkyl, aryl, and heteroaryl; or
R⁷ and R^{7′} or R⁹ and R^{9′}, together with the nitrogen atom to which they are attached, form a 4, 5, 6, or 7-membered heterocyclic ring;
R⁸ is alkyl, cycloalkyl, haloalkyl, aryl, or heteroaryl;
R¹⁰ is H, alkyl, haloalkyl, cycloalkyl, aryl, or heteroaryl;
R¹¹ is alkyl, aryl, or heteroaryl;
R¹² is H or alkyl;
p is an integer from 2 to 8;
PG is —PO₃H₂ or —SO₂NR^{m}Rⁿ;
R^{m} and Rⁿ are independently H, alkyl, or aryl; or R^{m} and Rⁿ, together with the nitrogen atom to which they are attached, form a 4, 5, 6, or 7-membered heterocyclic ring;
PDG is a prodrug moiety (PM) or a targeting moiety (TM), wherein said PDG group is connected to the group to which it is attached through a carbamate bond, an ester bond, or an ether bond.

2. The compound of claim 1, wherein said 4, 5, 6, or 7-membered heterocyclic ring formed by R⁷ and R^{7′} or R⁹ and R^{9′} or R^{m} and Rⁿ is 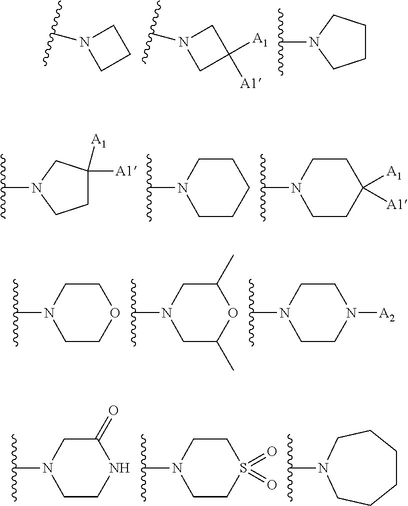
wherein
A₁ and A1′ are each H, F, or CH₃;
A₂ is H, —CH₃, -Et, -nPr, -iPr, —C(O)—CH₃, —S(O)₂—CH₃, —S(O)₂-Et, —S(O)₂-nPr, or —S(O)₂-iPr.

3. The compound of claim 1, wherein said targeting moiety (TM) is an antibody, a folate receptor binding moiety, a peptide, a nanoparticle based delivery vehicle, a selective tyrosine kinase inhibitor, or an Hsp90 inhibitor.

4. The compound of claim 1, wherein said targeting moiety (TM) is a targeting moiety connected through a nitrogen atom (—N(TM)) or an oxygen atom (—O(TM)) of the targeting moiety.

5. The compound of claim 1, wherein said PDG is represented by Formula 2, Formula 3, or Formula 4 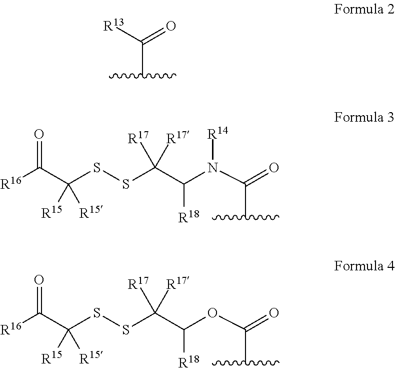
wherein
R¹³ is alkyl, haloalkyl, cycloalkyl, heterocycloalkyl, —NR⁹R^{9′}, —O-alkyl, or -(TM);
R¹⁴ is H, alkyl, haloalkyl, or cycloalkyl, wherein said alkyl, haloalkyl, and cycloalkyl contain 5-10 carbon atoms;
R¹⁵, R^{15′}, R¹⁷, or R^{17′} are each independently H, alkyl, or haloalkyl; or R¹⁵ and R^{15′} or R¹⁷ and R^{17′}, together with the carbon atom to which they are attached, form a 3, 4, 5-, or 6-membered heterocyclic ring;
R¹⁶ is —NR⁹R^{9′}, -TM, alkyl, haloalkyl, cycloalkyl, aryl, or heteroaryl; and
R¹⁸ is H or alkyl.

6. The compound of claim 1, wherein said compound of Formula 1 is represented by Formula 5a or its oxidized quinone form of Formula 5b 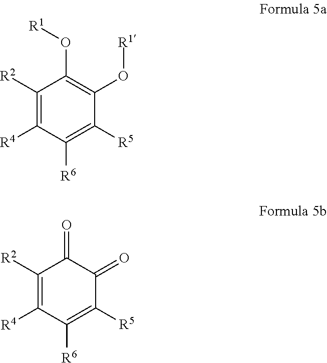
wherein
R⁶ is H, halogen, alkyl, haloalkyl, or cycloalkyl;
R^{1′} is H or PDG; or
R¹ and R^{1′}, together with the oxygen atoms to which they are attached, form a heterocyclic group represented by

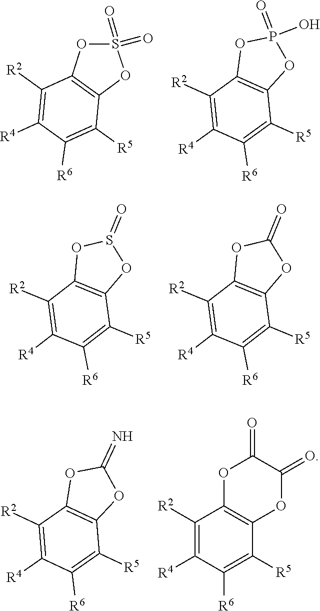

7. The compound of claim 1, wherein said compound of Formula 1 is represented by Formula 6a or its oxidized quinone form of Formula 6b 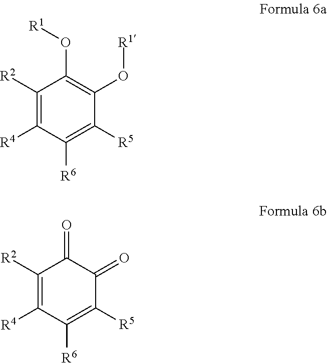
wherein
R⁵ is H, halogen, alkyl, haloalkyl, or cycloalkyl;
R^{1′} is H or PDG; or
R¹ and R^{1′}, together with the oxygen atoms to which they are attached, form a heterocyclic group represented by:

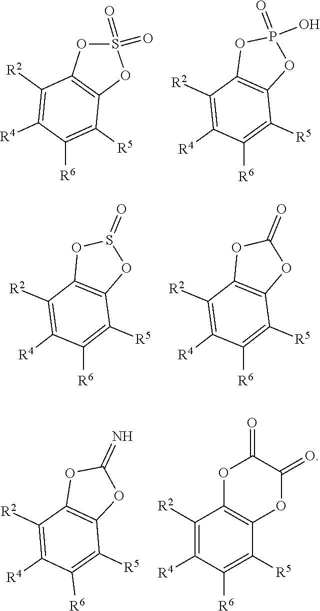

8. The compound of claim 1, wherein said compound of Formula 1 is represented by Formula 7a or its oxidized quinone form Formula 7b 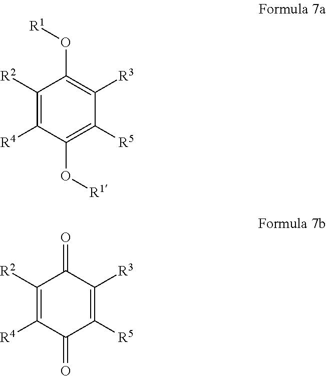
wherein R^{1′} is H or PDG.

9. The compound of claim 1, wherein said compound of Formula 1 is represented by Formula 8a or its oxidized quinone form Formula 8b 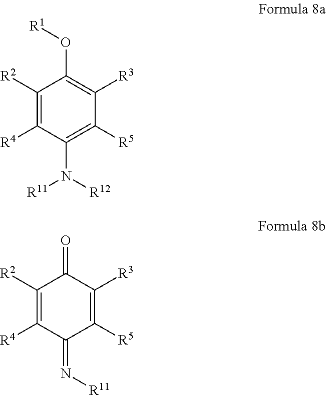
wherein
R¹¹ is aryl or heteroaryl, represented by the formulae: 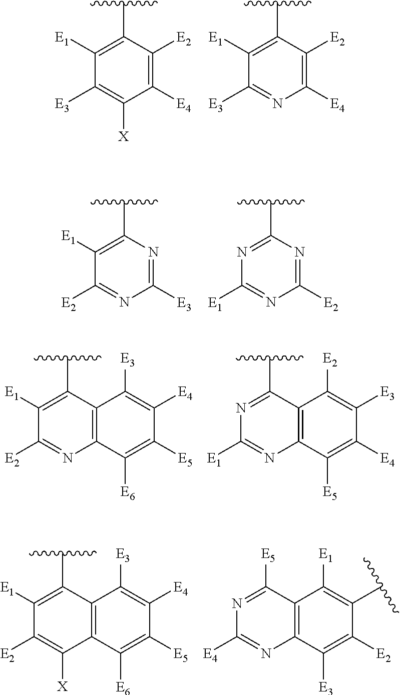
wherein
X is —O-alkyl or —NR^{m}Rⁿ;
E₁ to E₆ are each independently halo, —CH₃, -Et, —OCH₃, —OEt, or —CF₃;
R¹² is H; and
R^{m} and Rⁿ are independently H, alkyl, or aryl; or R^{m} and Rⁿ, together with the nitrogen atom to which they are attached, form a 4, 5, 6, or 7-membered heterocyclic ring.

10. The compound of claim 9, wherein said 4, 5, 6, or 7-membered heterocyclic ring is 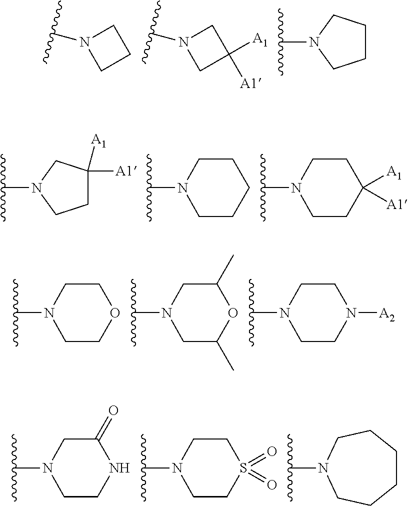
wherein
A₁ and A1′ are each H, F, or CH₃;
A₂ is H, —CH₃, -Et, -nPr, -iPr, —C(O)—CH₃, —S(O)₂—CH₃, —S(O)₂-Et, —S(O)₂-nPr, or —S(O)₂-iPr.

11-58. (canceled)

59. The compound of claim 1, wherein said compound is
N-(2-((3,4-dimethoxyphenyl)thio)ethyl)propionamide;
N-[2-(3,4-dihydroxyphenyl)sulfanylethyl]propenamide;
N-[2-(2,5-dihydroxyphenyl)sulfanylethyl]propenamide;
N-[2-(2,3-dihydroxyphenyl)sulfanylethyl]propenamide;
4-(2-aminoethylsulfanyl)benzene-1,2-diol;
N-[2-(3,4-dimethoxyphenyl) sulfanylethyl]heptanamide;
N-[2-(3,4-dihydroxyphenyl)sulfanylethyl]heptanamide;
N-[2-(3,4-dihydroxyphenyl)sulfanylethyl]undecanamide;
N-[2-(3,4-dimethoxyphenyl)sulfanylethyl]undecanamide;
N-[2-(3,4-dihydroxyphenyl)sulfanylethyl]undecanamide;
N-[2-(3,4-dimethoxyphenyl)sulfanylethyl]undecanamide;
N-[2-(3,4-dihydroxyphenyl)sulfanylethyl]pentanamide;
3-(5,5-difluoro-1,3-dimethyl-dipyrrolo[3,1-c:2′,1′-g][1,3,2]diazaborinin-4-ium-7-yl)-N-[2-(3,4-dihydroxyphenyl)sulfanylethyl]propenamide;
N-[2-(3,4-dihydroxyphenyl) sulfanylethyl]cyclopentanecarboxamide;
3-pentadecylbenzene-1,2-diol;
(4Z)-4-[(7-chloro-4-quinolyl)imino]-2-(diethylaminomethyl)cyclohexa-2,5-dien-1-one; or
3-[(Z)-pentadec-10-enyl]benzene-1,2-diol.

60. The compound of claim 1, wherein said conjugate is an antibody drug conjugate (ADC), a folate receptor drug conjugate (FRDC), a peptide drug conjugate, a nanoparticle containing liposomes, a polymer based vehicle, a hyaluronic acid based delivery vehicle, a conjugate with a selective tyrosine kinase inhibitor; or a conjugate with an Hsp90 inhibitor thereof.

61. A pharmaceutical composition comprising a compound of claim 1, or a pharmaceutically acceptable salt, conjugate, or prodrug thereof, and at least one pharmaceutically acceptable carrier.

62. A method for treating cancer, the method comprising the step of administering a therapeutically effective amount of a compound of claim 1, or a pharmaceutically acceptable salt, conjugate, or prodrug thereof, to a subject in need thereof, wherein said compound generates an immune response in situ at the tumor through a formation of neoantigen.

63. (canceled)

64. The method of claim 62, wherein the compound, or a pharmaceutically acceptable salt, conjugate, or prodrug thereof, is administered orally, intravenously, intraperitoneally, or intramuscularly.

65. (canceled)

66. (canceled)

67. The method of claim 62, wherein when dosed intratumorally, the compound has the form of hapten (protein reactive form), prohapten (requiring intracellular chemical transformation), or prodrugs of hapten or prohapten (requiring multiple intracellular transformation).

68. The method of claim 62, wherein said compound, or a pharmaceutically acceptable salt, conjugate, or prodrug thereof, is administered in combination with an anticancer drug.

69. The method of claim 68, wherein said anticancer drug is a cytotoxic drug selected from alkylating agents, antimitotic drugs, antimetabolites, topoisomerase 1 and 2 inhibitors, platinum drugs, anti-microtubule agents, and hormones, or derivatives thereof, a molecularly targeted agent, cell cycle signaling agent, modulators of tumor microenvironment, an antibody drug conjugate (ADC), radiation, an immunosuppressant, an enhancer of antigen presenting functions, an enhancer of T/macrophage effector, a drug that influences the purine metabolism, an agonist or antagonist of adenosine receptors, a proteasome inhibitor, a HDAC inhibitor, a TRAIL-R agonist, a chimeric antigen receptor t-cell therapy (CAR-T), an antitumor vaccine, or an immunomodulatory agent targeting PD-1, PD-L1, NKG2A, KIR, CTLA-4, LAG-3, TIM-3, BTLA, VISTA, PD-1H, TIGIT, CD96, STAT3, Arginase-1, HIF-1a, VEGF, CCL2, IDO, Tie2, CSF1, IL-10, IL-13, or IL-23, cancer vaccines, oncolytic virus, STING agonists, and TLR agonists.

70. The method of claim 62, wherein said cancer is a cancer that expresses an elevated level of reactive oxygen species.

71. The method of claim 62, wherein said cancer is lung cancer, breast cancer, liver cancer, leukemia, uroepithelial cancer, pancreatic cancer, ovarian cancer, colorectal cancer, head and neck cancer, melanoma, esophageal cancer, glioblastoma, prostate cancer, oral cancer, bladder cancer, gastric cancer, cervical cancer, or colon cancer.
